(19) United States
(12) Patent Application Publication
(10) Pub. No.: US 2024/0409523 A1
(43) Pub. Date: Dec. 12, 2024

(54) SUGAR AMIDES AND MIXTURES THEREOF

(71) Applicant: Clariant International Ltd, Muttenz (CH)

(72) Inventors: Soraya DELVOS, Burgkirchen (DE); Dirk LEINWEBER, Kelkheim (DE); Petra SCHAAL, Elnhausen (DE)

(21) Appl. No.: 18/695,096

(22) PCT Filed: Sep. 30, 2022
(86) PCT No.: PCT/EP2022/077344

§ 371 Date: Mar. 25, 2024

(30) Foreign Application Priority Data
Oct. 1, 2021 (EP) 21200582.1

(51) Int. Cl. C07D 307/20; A01N 43/08; A01P 1/00; A61K 8/49; A61Q 17/00

(52) U.S. Cl. CPC C07D 307/20; A01N 43/08; A01P 1/00; A61K 8/4973; A61Q 17/005

(57) ABSTRACT

The invention relates to sugar amides and mixtures thereof as well as their use in cosmetic or household applications and oil or mining applications.

The invention relates to sugar amides and mixtures thereof as well as their use in cosmetic or household applications and oil or mining applications.

Sugar amides have proven to be versatile ingredients for various applications. WO 2013/178683, WO 2014/170025, WO 2014/206554, WO 2014/206555, among others, describe N-alkyl N-acyl glucamines and their use in cosmetic and household formulations, for example as surfactants. WO 2016/146303 discloses cyclic N-alkyl glucamides and their use as surfactants in compositions for enhanced oil recovery. WO 2018/002100 discloses cyclic N-alkyl glucamides and their use as antimicrobial agents or preservative boosters. WO 2014/138141 discloses mixtures of linear sugar amide surfactants for use in cleaning products.

There is an ongoing need for versatile ingredients for various applications, including cosmetic or household applications and oil or mining applications. In particular, there is a need for ingredients, such as surfactants, that show good performance as well as are sustainable and environmentally friendly. Furthermore, there is a need for ingredients, such as surfactants, that show good formulatability and formulation flexibility.

Preservation of household formulations and cosmetic formulations extends their shelf life and therefore provides greater value for money for consumers. Furthermore, preservatives prevent consumers from distributing microbes around their home or on themselves and hence provide health benefits. There is a need for antimicrobial agents or preservative boosters that show good performance as well as are sustainable and environmentally friendly.

Many ingredients for use in cosmetic or household cleaning formulations are traditionally derived from crude oil. Environmental, economic and sustainability questions are restricting the use of products derived from this limited resource. Consumers have generally become more conscious about the ingredients used in, e.g., cosmetic products or household cleaning products. Among others, they desire sustainable and environmentally friendly ingredients that are derived from natural and renewable sources. Consumers perceive compounds derived from natural materials to be gentler and more environmentally friendly.

It has now been found that cyclic sugar amides show good antimicrobial properties. Accordingly, the present invention relates to a compound of Formula (I)

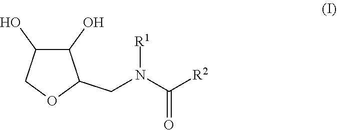

wherein
R¹ is selected from hydrogen, C1-C16 alkyl, C1-C3 hydroxyalkyl, and methoxy-(C1-C3)-alkyl;
R² is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl.

It has also been found that mixtures of sugar amides wherein at least one sugar amide is a cyclic sugar amide are versatile ingredients for various applications. Accordingly, the present invention also relates to a mixture comprising a first sugar amide and a second sugar amide, wherein the first sugar amide is a cyclic sugar amide.

Advantageously, the compounds of Formula (I) and the mixtures of the invention can be derived from natural and renewable sources (e.g. naturally occurring sugars and/or fatty acids derived from naturally occurring oils) and are thus sustainable and environmentally friendly. The compounds of Formula (I) and the mixtures of the invention have a high renewable carbon index (RCI). Furthermore, the compounds of Formula (I) and the mixtures of the invention show good performance and physical properties as well as good formulatability and formulation flexibility. The compounds of Formula (I) and the mixtures of the invention show good antimicrobial properties.

The mixture of the invention comprises a first sugar amide and a second sugar amide.

As used herein, the term “sugar amide” preferably refers to a sugar wherein one aldehyde group is replaced by an acylaminoalkyl group.

According to the invention, the first sugar amide is a cyclic sugar amide.

Preferably, the second sugar amide is a linear sugar amide or a cyclic sugar amide.

In a preferred embodiment, the second sugar amide is a linear sugar amide.

In another preferred embodiment, the second sugar amide is a cyclic sugar amide.

As used herein, the term “cyclic sugar amide” preferably refers to a cyclic sugar wherein one aldehyde group is replaced by an acylaminoalkyl group.

As used herein, the term “linear sugar amide” preferably refers to a linear sugar wherein one aldehyde group is replaced by an acylaminoalkyl group.

The first sugar amide can be derived from any sugar. Preferably, the first sugar amide is derived from a monosaccharide. More preferably, the first sugar amide is derived from glucose, xylose, arabinose, galactose, or mannose. Even more preferably, the first sugar amide is derived from glucose or xylose.

Particularly preferably, the first sugar amide is derived from glucose. Also particularly preferably, the first sugar amide is derived from xylose.

The second sugar amide can be derived from any sugar. Preferably, the second sugar amide is derived from a monosaccharide. More preferably, the second sugar amide is derived from glucose, xylose, arabinose, galactose, or mannose. Even more preferably, the second sugar amide is derived from glucose or xylose. Particularly preferably, the second sugar amide is derived from glucose. Also particularly preferably, the second sugar amide is derived from xylose.

Preferably, the first sugar amide is derived from glucose, xylose, arabinose, galactose, or mannose, and the second sugar amide is derived from glucose, xylose, arabinose, galactose, or mannose. More preferably, the first sugar amide is derived from glucose or xylose, and the second sugar amide is derived from glucose or xylose.

In a particularly preferred embodiment, the first sugar amide is derived from glucose, and the second sugar amide is derived from glucose. In a particularly preferred embodiment, the first sugar amide is derived from glucose, and the second sugar amide is derived from xylose. In a particularly preferred embodiment, the first sugar amide is derived from xylose, and the second sugar amide is derived from glucose. In a particularly preferred embodiment, the first sugar amide is derived from xylose, and the second sugar amide is derived from xylose.

In a preferred embodiment, the second sugar amide is a linear sugar amide derived from glucose, xylose, arabinose, galactose, or mannose, more preferably a linear sugar amide derived from glucose or xylose, particularly preferably a linear sugar amide derived from glucose, also particularly preferably a linear sugar amide derived from xylose.

In another preferred embodiment, the second sugar amide is a cyclic sugar amide derived from glucose, xylose, arabinose, galactose, or mannose, more preferably a cyclic sugar amide derived from glucose or xylose, particularly preferably a cyclic sugar amide derived from glucose, also particularly preferably a cyclic sugar amide derived from xylose.

In a particularly preferred embodiment, the first sugar amide is derived from glucose, and the second sugar amide is a linear sugar amide derived from glucose.

In a particularly preferred embodiment, the first sugar amide is derived from glucose, and the second sugar amide is a cyclic sugar amide derived from glucose.

In a particularly preferred embodiment, the first sugar amide is derived from glucose, and the second sugar amide is a linear sugar amide derived from xylose.

In a particularly preferred embodiment, the first sugar amide is derived from glucose, and the second sugar amide is a cyclic sugar amide derived from xylose.

In a particularly preferred embodiment, the first sugar amide is derived from xylose, and the second sugar amide is a linear sugar amide derived from glucose.

In a particularly preferred embodiment, the first sugar amide is derived from xylose, and the second sugar amide is a cyclic sugar amide derived from glucose.

In a particularly preferred embodiment, the first sugar amide is derived from xylose, and the second sugar amide is a linear sugar amide derived from xylose.

In a particularly preferred embodiment, the first sugar amide is derived from xylose, and the second sugar amide is a cyclic sugar amide derived from xylose.

In a preferred embodiment, the cyclic sugar amide is selected from:
a compound of Formula (I)

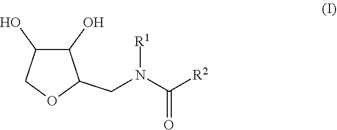

wherein
R¹ is selected from hydrogen, C1-C16 alkyl, C1-C3 hydroxyalkyl, and methoxy-(C1-C3)-alkyl;
R² is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
a compound of Formula (II)

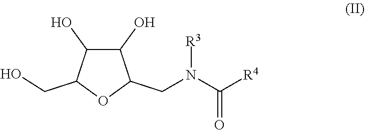

wherein
R³ is selected from hydrogen, C1-C16 alkyl, C1-C3 hydroxyalkyl, and methoxy-(C1-C3)-alkyl;
R⁴ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
a compound of Formula (III)

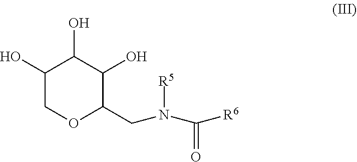

wherein
R⁵ is selected from hydrogen, C1-C16 alkyl, C1-C3 hydroxyalkyl, and methoxy-(C1-C3)-alkyl;
R⁶ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
and
a compound of Formula (IV)

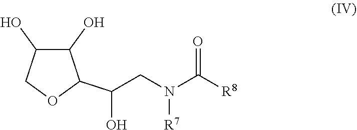

wherein
R⁷ is selected from hydrogen, C1-C16 alkyl, C1-C3 hydroxyalkyl, and methoxy-(C1-C3)-alkyl;
R⁸ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl.

In a more preferred embodiment, the cyclic sugar amide is selected from:
a compound of Formula (I)

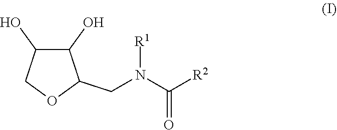

wherein
R¹ is selected from hydrogen, C1-C16 alkyl, C1-C3 hydroxyalkyl, and methoxy-(C1-C3)-alkyl;
R² is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
and
a compound of Formula (II)

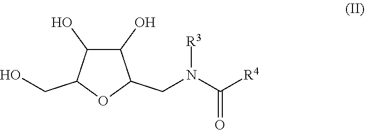

wherein
R³ is selected from hydrogen, C1-C16 alkyl, C1-C3 hydroxyalkyl, and methoxy-(C1-C3)-alkyl;
R⁴ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl.

Preferred are compounds of Formula (I) wherein
R¹ is selected from C1-C4 alkyl;
R² is selected from C7-C21 alkyl, C7-C21 monoalkenyl, and C7-C21 dialkenyl.

More preferred are compounds of Formula (I) wherein
R¹ is methyl or ethyl, preferably methyl;
R² is selected from C7-C17 alkyl and C7-C17 monoalkenyl, preferably is selected from C7-C17 alkyl.

Preferred are compounds of Formula (II) wherein
R³ is selected from C1-C4 alkyl;
R⁴ is selected from C7-C21 alkyl, C7-C21 monoalkenyl, and C7-C21 dialkenyl.

More preferred are compounds of Formula (II) wherein
R³ is methyl or ethyl, preferably methyl;
R⁴ is selected from C7-C17 alkyl and C7-C17 monoalkenyl, preferably is selected from C7-C17 alkyl.

Preferred are compounds of Formula (III) wherein
R⁵ is selected from C1-C4 alkyl;
R⁶ is selected from C7-C21 alkyl, C7-C21 monoalkenyl, and C7-C21 dialkenyl.

More preferred are compounds of Formula (III) wherein
R⁵ is methyl or ethyl, preferably methyl;
R⁶ is selected from C7-C17 alkyl and C7-C17 monoalkenyl, preferably is selected from C7-C17 alkyl.

Preferred are compounds of Formula (IV) wherein
R⁷ is selected from C1-C4 alkyl;
R⁸ is selected from C7-C21 alkyl, C7-C21 monoalkenyl, and C7-C21 dialkenyl.

More preferred are compounds of Formula (IV) wherein
R⁷ is methyl or ethyl, preferably methyl;
R⁸ is selected from C7-C17 alkyl and C7-C17 monoalkenyl, preferably is selected from C7-C17 alkyl.

Compounds of Formula (I) can, for example, be prepared by a process comprising reacting an N-alkyl-xylamine with one or more carboxylic acids R²—COOH or one or more carboxylic esters R²—COO(C1-C2 alkyl) or one or more triglycerides. Carboxylic acids R²—COOH or triglycerides are particularly preferred.

Typically, R² in the “one or more carboxylic acids R²—COOH” is defined as R² in Formula (I). Typically, R² in the “one or more carboxylic esters R²—COO(C1-C2 alkyl)” is defined as R² in Formula (I). Typically, the fatty acid residue(s) in the triglycerides are as the fatty acid residue(s) in Formula (I).

Preferably, the N-alkyl-xylamine is an N—(C1-C4 alkyl)-xylamine, more preferably N-methyl-xylamine or N-ethyl-xylamine, particularly preferably N-methyl-xylamine.

N-methyl-xylamine can be prepared by methods known in the art, for example as described in WO 2014/138141. Carboxylic acids R²—COOH or carboxylic esters R²—COO(C1-C2 alkyl) or triglycerides are commercially available or can be prepared by methods known in the art.

In one embodiment, the N-alkyl-xylamine is reacted with the one or more carboxylic acids R²—COOH or one or more carboxylic esters R²—COO(C1-C2 alkyl) or one or more triglycerides in the presence of a catalyst. Preferred catalysts are selected from phosphonic acid, phosphoric acid, methane sulfonic acid, p-toluene sulfonic acid, alkylbenzene sulfonic acid, sulfuric acid, diethylphosphite, and mixtures of phosphonic acid and sodium hydroxide. A particularly preferred catalyst is phosphonic acid.

Typically, water is boiled off during the reaction of the N-alkyl-xylamine with the one or more carboxylic acids R²—COOH or one or more carboxylic esters R²—COO(C1-C2 alkyl) or one or more triglycerides.

In one embodiment, the N-alkyl-xylamine is reacted with the one or more carboxylic acids R²—COOH or one or more carboxylic esters R²—COO(C1-C2 alkyl) or one or more triglycerides at a temperature of at least 100° C., preferably at least 120° C., more preferably at least 130° C., even more preferably at least 140° C., even more preferably at least 150° C., particularly preferably from 150° C. to 170° C., for example 160° C.

In one embodiment, the N-alkyl-xylamine is reacted with the one or more carboxylic acids R²—COOH or one or more carboxylic esters R²—COO(C1-C2 alkyl) or one or more triglycerides at ambient pressure (1013 mbar). In another embodiment, the N-alkyl-xylamine is reacted with the one or more carboxylic acids R²—COOH or one or more carboxylic esters R²—COO(C1-C2 alkyl) or one or more triglycerides at a pressure of from 0 mbar to 200 mbar, preferably from 10 mbar to 100 mbar, more preferably from 20 mbar to 75 mbar, particularly preferably from 30 mbar to 60 mbar, for example 50 mbar. In another embodiment, the N-alkyl-xylamine is reacted with the one or more carboxylic acids R²—COOH or one or more carboxylic esters R²—COO(C1-C2 alkyl) or one or more triglycerides at a pressure of from 200 mbar to 1013 mbar. In a particular embodiment, the reaction of the N-alkyl-xylamine with the one or more carboxylic acids R²—COOH or one or more carboxylic esters R²—COO(C1-C2 alkyl) or one or more triglycerides is first carried out at ambient pressure (1013 mbar) and then at a pressure of from 0 mbar to 200 mbar, preferably from 10 mbar to 100 mbar, more preferably from 20 mbar to 75 mbar, particularly preferably from 30 mbar to 60 mbar, for example 50 mbar.

In one embodiment, the N-alkyl-xylamine is reacted with the one or more carboxylic acids R²—COOH or one or more carboxylic esters R²—COO(C1-C2 alkyl) or one or more triglycerides for at least 2 hours, preferably at least 3 hours, more preferably at least 4 hours, particularly preferably at least 6 hours. Preferably, the N-alkyl-xylamine is reacted with the one or more carboxylic acids R²—COOH or one or more carboxylic esters R²—COO(C1-C2 alkyl) or one or more triglycerides for 4 to 12 hours, particularly preferably 6 to 12 hours.

Typically, the N-alkyl-xylamine is reacted with the one or more carboxylic acids R²—COOH or one or more carboxylic esters R²—COO(C1-C2 alkyl) or one or more triglycerides under nitrogen gas.

Compounds of Formula (I) can, for example, also be prepared from compounds of Formula (VI), for example by reacting the latter with carboxylic acids R²—COOH or derivatives thereof or triglycerides.

Compounds that fall within the scope of Formula (II) are described in WO 2018/002100 und WO 2016/146303. A product having the INCI name Capryloyl/Caproyl Anhydro Methyl Glucamide (and) Water is commercially available from Clariant (Velsan® Flex).

Compounds of Formula (II) can, for example, be prepared as described in WO 2018/002100.

Compounds of Formula (II) can, for example, be prepared by a process comprising reacting an N-alkyl-glucamine with one or more carboxylic acids R⁴—COOH or one or more carboxylic esters R⁴—COO(C1-C2 alkyl) or one or more triglycerides. Carboxylic acids R⁴—COOH or triglycerides are particularly preferred.

Typically, R⁴ in the “one or more carboxylic acids R⁴—COOH” is defined as R⁴ in Formula (II). Typically, R⁴ in the “one or more carboxylic esters R⁴—COO(C1-C2 alkyl)” is defined as R⁴ in Formula (II). Typically, the fatty acid residue(s) in the triglycerides are as the fatty acid residue(s) in Formula (II).

Preferably, the N-alkyl-glucamine is an N—(C1-C4 alkyl)-glucamine, more preferably N-methyl-glucamine or N-ethyl-glucamine, particularly preferably N-methyl-glucamine.

N-methyl-glucamine is commercially available (e.g. from Sigma-Aldrich) or can be prepared by methods known in the art. N-ethyl-glucamine is commercially available (e.g. from Sigma Aldrich) or can be prepared by methods known in the art. Carboxylic acids R⁴—COOH or carboxylic esters R⁴—COO(C1-C2 alkyl) or triglycerides are commercially available or can be prepared by methods known in the art.

In one embodiment, the N-alkyl-glucamine is reacted with the one or more carboxylic acids R⁴—COOH or one or more carboxylic esters R⁴—COO(C1-C2 alkyl) or one or more triglycerides in the presence of a catalyst. Preferred catalysts are selected from phosphonic acid, phosphoric acid, methane sulfonic acid, p-toluene sulfonic acid, alkylbenzene sulfonic acid, sulfuric acid, diethylphosphite, and mixtures of phosphonic acid and sodium hydroxide. A particularly preferred catalyst is phosphonic acid.

Typically, water is boiled off during the reaction of the N-alkyl-glucamine with the one or more carboxylic acids R⁴—COOH or one or more carboxylic esters R⁴—COO(C1-C2 alkyl) or one or more triglycerides.

In one embodiment, the N-alkyl-glucamine is reacted with the one or more carboxylic acids R⁴—COOH or one or more carboxylic esters R⁴—COO(C1-C2 alkyl) or one or more triglycerides at a temperature of at least 100° C., preferably at least 120° C., more preferably at least 130° C., even more preferably at least 140° C., even more preferably at least 150° C., particularly preferably from 150° C. to 170° C., for example 160° C.

In one embodiment, the N-alkyl-glucamine is reacted with the one or more carboxylic acids R⁴—COOH or one or more carboxylic esters R⁴—COO(C1-C2 alkyl) or one or more triglycerides at ambient pressure (1013 mbar). In another embodiment, the N-alkyl-glucamine is reacted with the one or more carboxylic acids R⁴—COOH or one or more carboxylic esters R⁴—COO(C1-C2 alkyl) or one or more triglycerides at a pressure of from 0 mbar to 200 mbar, preferably from 10 mbar to 100 mbar, more preferably from 20 mbar to 75 mbar, particularly preferably from 30 mbar to 60 mbar, for example 50 mbar. In another embodiment, the N-alkyl-glucamine is reacted with the one or more carboxylic acids R⁴—COOH or one or more carboxylic esters R⁴—COO(C1-C2 alkyl) or one or more triglycerides at a pressure of from 200 mbar to 1013 mbar. In a particular embodiment, the reaction of the N-alkyl-glucamine with the one or more carboxylic acids R⁴—COOH or one or more carboxylic esters R⁴—COO(C1-C2 alkyl) or one or more triglycerides is first carried out at ambient pressure (1013 mbar) and then at a pressure of from 0 mbar to 200 mbar, preferably from 10 mbar to 100 mbar, more preferably from 20 mbar to 75 mbar, particularly preferably from 30 mbar to 60 mbar, for example 50 mbar.

In one embodiment, the N-alkyl-glucamine is reacted with the one or more carboxylic acids R⁴—COOH or one or more carboxylic esters R⁴—COO(C1-C2 alkyl) or one or more triglycerides for at least 2 hours, preferably at least 3 hours, more preferably at least 4 hours, particularly preferably at least 6 hours. Preferably, the N-alkyl-glucamine is reacted with the one or more carboxylic acids R⁴—COOH or one or more carboxylic esters R⁴—COO(C1-C2 alkyl) or one or more triglycerides for 4 to 12 hours, particularly preferably 6 to 12 hours.

Typically, the N-alkyl-glucamine is reacted with the one or more carboxylic acids R⁴—COOH or one or more carboxylic esters R⁴—COO(C1-C2 alkyl) or one or more triglycerides under nitrogen gas.

Compounds that fall within the scope of Formula (III) are described in WO 2018/002100 und WO 2016/146303.

Compounds that fall within the scope of Formula (IV) are described in WO 2018/002100 und WO 2016/146303.

In a preferred embodiment, the linear sugar amide is a compound of Formula (V)

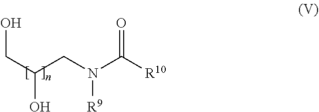

wherein
R⁹ is selected from hydrogen, C1-C16 alkyl, C1-C3 hydroxyalkyl, and methoxy-(C1-C3)-alkyl;
R¹⁰ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
n is selected from 1 to 4.

Preferred are compounds of Formula (V) wherein
R⁹ is selected from C1-C4 alkyl;
R¹⁰ is selected from C7-C21 alkyl, C7-C21 monoalkenyl, and C7-C21 dialkenyl;
n is 3 or 4.

More preferred are compounds of Formula (V) wherein
R⁹ is methyl or ethyl, preferably methyl;
R¹⁰ is selected from C7-C17 alkyl and C7-C17 monoalkenyl, preferably is selected from C7-C17 alkyl;
n is 3 or 4.

Compounds that fall within the scope of Formula (V) are described in e.g. WO 2013/178700. N-methyl glucamides are commercially available from Clariant (GlucoTain®, GlucoPure®).

Compounds of Formula (V) can, for example, be prepared as described in e.g. WO 2013/178700. Compounds of Formula (V) can, for example, be prepared by reacting N-alkyl sugar amines with fatty acid esters or triglycerides.

In a preferred embodiment, the first sugar amide is selected from a compound of formula (I), a compound of formula (II), a compound of formula (III), and a compound of formula (IV).

In a preferred embodiment, the second sugar amide is selected from a compound of formula (I), a compound of formula (II), a compound of formula (III), a compound of formula (IV), and a compound of formula (V).

In a preferred embodiment, the second sugar amide is selected from a compound of formula (I), a compound of formula (II), a compound of formula (III), and a compound of formula (IV).

In a preferred embodiment, the second sugar amide is a compound of formula (V).

In a preferred embodiment, the first sugar amide is a compound of Formula (I)

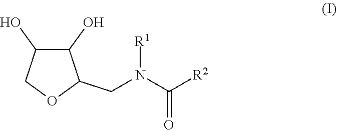

wherein
R¹ is selected from hydrogen, C1-C16 alkyl, C1-C3 hydroxyalkyl, and methoxy-(C1-C3)-alkyl;
R² is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
and the second sugar amide is a compound of Formula (V)

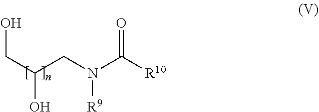

wherein
R⁹ is selected from hydrogen, C1-C16 alkyl, C1-C3 hydroxyalkyl, and methoxy-(C1-C3)-alkyl;
R¹⁰ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
n is selected from 1 to 4.

In a preferred embodiment, the first sugar amide is a compound of Formula (II)

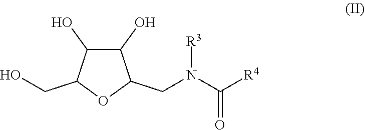

wherein
R³ is selected from hydrogen, C1-C16 alkyl, C1-C3 hydroxyalkyl, and methoxy-(C1-C3)-alkyl;
R⁴ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
and the second sugar amide is a compound of Formula (V)

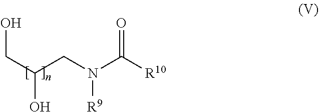

wherein
R⁹ is selected from hydrogen, C1-C16 alkyl, C1-C3 hydroxyalkyl, and methoxy-(C1-C3)-alkyl;
R¹⁰ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
n is selected from 1 to 4.

In a preferred embodiment, the first sugar amide is a compound of Formula (I)

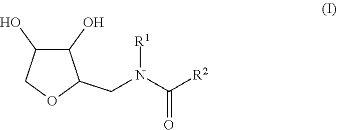

wherein
R¹ is selected from hydrogen, C1-C16 alkyl, C1-C3 hydroxyalkyl, and methoxy-(C1-C3)-alkyl;
R² is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
and the second sugar amide is a compound of Formula (II)

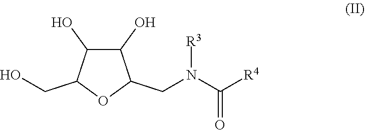

wherein
R³ is selected from hydrogen, C1-C16 alkyl, C1-C3 hydroxyalkyl, and methoxy-(C1-C3)-alkyl;
R⁴ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl.

Preferably, the weight ratio of the first sugar amide to the second sugar amide is from 99.5:0.5 to 0.5:99.5, or from 99:1 to 1:99, or from 95:5 to 5:95, or from 90:10 to 10:90, or from 80:20 to 20:80, or from 75:25 to 25:75, or from 70:30 to 30:70, or from 60:40 to 40:60, or from 55:45 to 45:55.

In some embodiments, the weight ratio of the first sugar amide to the second sugar amide is from 50:50 to 99:1, or from 60:40 to 90:10, or from 70:30 to 80:20.

In some embodiments, the weight ratio of the first sugar amide to the second sugar amide is from 1:99 to 50:50, or from 10:90 to 40:60, or from 20:80 to 30:70.

In preferred embodiments, the mixture of the invention comprises
from 1 to 99 wt.-%, preferably from 10 to 90 wt.-%, more preferably from 20 to 80 wt.-%, even more preferably from 30 to 70 wt.-%, particularly preferably from 40 to 60 wt.-% of the first sugar amide, based on the total weight of the mixture, and
from 1 to 99 wt.-%, preferably from 10 to 90 wt.-%, more preferably from 20 to 80 wt.-%, even more preferably from 30 to 70 wt.-%, particularly preferably from 40 to 60 wt.-% of the second sugar amide, based on the total weight of the mixture.

In some embodiments, the mixture of the invention comprises
from 50 to 99 wt.-%, preferably from 60 to 90 wt.-%, particularly preferably from 70 to 80 wt.-% of the first sugar amide, based on the total weight of the mixture, and
from 1 to 50 wt.-%, preferably from 10 to 40 wt.-%, particularly preferably from 20 to 30 wt.-% of the second sugar amide, based on the total weight of the mixture.

In some embodiments, the mixture of the invention comprises
from 1 to 50 wt.-%, preferably from 10 to 40 wt.-%, particularly preferably from 20 to 30 wt.-% of the first sugar amide, based on the total weight of the mixture, and
from 50 to 99 wt.-%, preferably from 60 to 90 wt.-%, particularly preferably from 70 to 80 wt.-% of the second sugar amide, based on the total weight of the mixture.

The mixture of the invention may comprise further components.

Optionally, the mixture of the invention comprises further sugar amides. In one embodiment, the mixture of the invention further comprises a third sugar amide. In one embodiment, the mixture of the invention further comprises a third sugar amide and a fourth sugar amide. In one embodiment, the mixture of the invention further comprises a third sugar amide, a fourth sugar amide, and a fifth sugar amide.

Preferably, such further sugar amides are linear sugar amides or cyclic sugar amides.

In one embodiment, the third sugar amide, if present, is a linear sugar amide.

In another embodiment, the third sugar amide, if present, is a cyclic sugar amide.

In one embodiment, the fourth sugar amide, if present, is a linear sugar amide.

In another embodiment, the fourth sugar amide, if present, is a cyclic sugar amide.

In one embodiment, the fifth sugar amide, if present, is a linear sugar amide.

In another embodiment, the fifth sugar amide, if present, is a cyclic sugar amide.

Such further sugar amides can be derived from any sugar. Preferably, such further sugar amides are derived from a monosaccharide. More preferably, such further sugar amides are derived from glucose, xylose, arabinose, galactose, and/or mannose.

Preferably, such further sugar amides are selected from a compound of formula (I), a compound of formula (II), a compound of formula (III), a compound of formula (IV), and a compound of formula (V).

In one embodiment, the third sugar amide, if present, is selected from a compound of formula (I), a compound of formula (II), a compound of formula (III), a compound of formula (IV), and a compound of formula (V).

In one embodiment, the fourth sugar amide, if present, is selected from a compound of formula (I), a compound of formula (II), a compound of formula (III), a compound of formula (IV), and a compound of formula (V).

In one embodiment, the fifth sugar amide, if present, is selected from a compound of formula (I), a compound of formula (II), a compound of formula (III), a compound of formula (IV), and a compound of formula (V).

Optionally, the mixture of the invention further comprises one or more esters of sugar amides. As used herein, the term “ester of a sugar amide” refers to a sugar amide as defined herein wherein one or more hydroxy groups are replaced by an acyloxy group.

Optionally, the mixture of the invention further comprises a solvent. In at least one embodiment, the solvent comprises water and/or alcohol. In at least one embodiment, the solvent is cosmetically acceptable. In a particularly preferred embodiment, the solvent is water. Water is useful for economic reasons but also because it is cosmetically acceptable. Optionally, the mixture of the invention comprises water-miscible or water-soluble solvents, such as C1-C5 alkyl monohydric alcohols, preferably C2-C3 alkyl monohydric alcohols.

In a preferred embodiment, the mixture of the invention comprises a solvent selected from the group consisting of water, glycols, ethanol, and mixtures thereof. In a particularly preferred embodiment, the mixture comprises water as the solvent.

In a preferred embodiment, the mixture of the invention comprises an aqueous, alcoholic or aqueous-alcoholic solvent, wherein the aqueous, alcoholic or aqueous-alcoholic solvent comprises water, ethanol, propanol, isopropanol, 1,2-propylene glycol, 1,3-propylene glycol, isobutanol, butanol, butyl glycol, butyl diglycol, glycerol, or mixtures thereof; preferably wherein the aqueous, alcoholic or aqueous-alcoholic solvent comprises water, ethanol, propanol, isopropanol, 1,2-propylene glycol, 1,3-propylene glycol, glycerol, or mixtures thereof; more preferably wherein the aqueous, alcoholic or aqueous-alcoholic solvent comprises water, isopropanol, 1,2-propylene glycol, 1,3-propylene glycol, or mixtures thereof; even more preferably wherein the aqueous, alcoholic or aqueous-alcoholic solvent consists of water or consists of a mixture of water and an alcohol wherein the alcohol is selected from the group consisting of isopropanol, 1,2-propylene glycol and 1,3-propylene glycol.

The mixture of the invention can, for example, be prepared by mixing its ingredients.

The mixture of the invention may, for example, be used to prepare a formulation, preferably a formulation of the invention. Such a formulation may, for example, be a cosmetic formulation or a household cleaning formulation. Such a formulation may, for example, also be a formulation for oil or mining applications. Such a formulation may, for example, also be a formulation for technical or industrial applications.

The invention also relates to a compound of Formula (I)

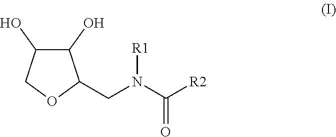

wherein
R¹ is selected from hydrogen, C1-C16 alkyl, C1-C3 hydroxyalkyl, and methoxy-(C1-C3)-alkyl;
R² is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl.

Preferred are compounds of Formula (I) wherein
R¹ is selected from C1-C4 alkyl;
R² is selected from C7-C21 alkyl, C7-C21 monoalkenyl, and C7-C21 dialkenyl.

More preferred are compounds of Formula (I) wherein
R¹ is methyl or ethyl, preferably methyl;
R² is selected from C7-C17 alkyl and C7-C17 monoalkenyl, preferably is selected from C7-C17 alkyl.

The compound of Formula (I) may, for example, be used to prepare a mixture of the invention.

Advantageously, the compounds of Formula (I) can be derived from natural and renewable sources (e.g. naturally occurring sugars and/or fatty acids derived from naturally occurring oils) and are thus sustainable and environmentally friendly. The compounds of Formula (I) have a high renewable carbon index (RCI). Furthermore, the compounds of Formula (I) show good performance and physical properties as well as good formulatability and formulation flexibility. The compounds of Formula (I) and the mixtures of the invention show good antimicrobial properties. Advantageously, the compounds of Formula (I) are soluble in water and stable over a wide pH range.

The compound of Formula (I) is based on xylose, also called wood sugar, and derived from hemicellulose such as sawdust or rice straw. In comparison to compounds which are derived from corn or wheat as sugar backbone, the compound of Formula (I) does not compete with these food sources. This enables use in formulations with high renewable content without competing with food applications.

The compound of Formula (I) may, for example, be used to prepare a formulation, preferably a formulation of the invention. Such a formulation may, for example, be a cosmetic formulation or a household cleaning formulation. Such a formulation may, for example, also be a formulation for oil or mining applications. Such a formulation may, for example, also be a formulation for technical or industrial applications.

The invention also relates to a compound of Formula (VI)

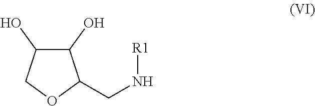

wherein
R¹ is selected from hydrogen, C1-C16 alkyl, C1-C3 hydroxyalkyl, and methoxy-(C1-C3)-alkyl.

Preferred are compounds of Formula (VI) wherein
R¹ is selected from C1-C4 alkyl.

More preferred are compounds of Formula (VI) wherein
R¹ is methyl or ethyl, preferably methyl.

The compound of Formula (VI) may, for example, be used to prepare a compound of Formula (I).

The present invention also relates to an antimicrobial blend comprising
(A) a compound of Formula (I) of the present invention, as component A; and
(B) an antimicrobial agent or a preservative booster, as component B.

In at least one embodiment, the weight ratio of component A to component B is from 1:20 to 20:1.

The antimicrobial blend of the present invention comprises an antimicrobial agent or a preservative booster, as component B.

In at least one embodiment, the antimicrobial blend of the present invention comprises an antimicrobial agent.

In at least one embodiment, the antimicrobial agent is selected from the group consisting of aromatic alcohols, organic acids and salts thereof, hydroxamic acids and salts thereof, compounds according to Formula (P), alkyl diols, halogenated compounds, isothiazolinones, and mixtures thereof; wherein Formula (P) is as follows:

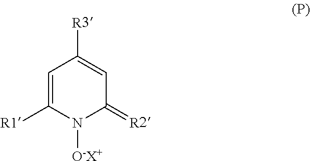

wherein
R1′ is H, an unsubstituted or halogen-substituted, branched or unbranched C₁-C₂₀-alkyl radical, an unsubstituted or halogen-substituted C₅-C₈-cycloalkyl radical, an unsubstituted or halogen-substituted C₆-C₁₀-aryl radical or an unsubstituted or halogen-substituted, branched or unbranched C₇-C₂₀-aralkyl radical;
R2′ is O or S;
R3′ is H or a C₁-C₄-alkyl radical;
X⁺ is a cation.

Preferably, R3′ is methyl.

In at least one embodiment, the antimicrobial agent is selected from the group consisting of aromatic alcohols, organic acids and salts thereof, hydroxamic acids and salts thereof, hydroxypyridones, alkyl diols, halogenated compounds, isothiazolinones, and mixtures thereof.

In at least one embodiment, the aromatic alcohols are selected from the group consisting of phenoxyethanol, benzyl alcohol, veratryl alcohol, propylene phenoxyethanol, phenethyl alcohol, phenylpropanol, vanillin, 2-methyl-1-phenyl-2-propanol, hydroxyethoxyphenyl butanone, methylparaben, ethylparaben, propylparaben, and mixtures thereof.

In at least one embodiment, the organic acids and salts thereof are selected from the group consisting of benzoic acid, sorbic acid, dehydroacetic acid, lactic acid, salicylic acid, p-anisic acid, undecylenic acid, glycolic acid, propionic acid, levulinic acid, and mixtures thereof.

In at least one embodiment, the hydroxamic acid is selected from hydroxamic acids of Formula (VII)

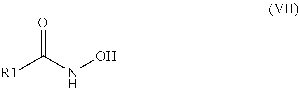

wherein R¹ is selected from saturated hydrocarbon chains having 5 to 23 carbon atoms, unsaturated hydrocarbon chains having 5 to 23 carbon atoms, and mixtures thereof.

Preferably, R¹ in Formula (VII) is selected from saturated hydrocarbon chains having 5 to 17 carbon atoms, unsaturated hydrocarbon chains having 5 to 17 carbon atoms, and mixtures thereof. More preferably, R¹ in Formula (VII) is selected from saturated hydrocarbon chains having 5 to 13 carbon atoms, unsaturated hydrocarbon chains having 5 to 13 carbon atoms, and mixtures thereof. Particularly preferably, R¹ in Formula (VII) is selected from saturated hydrocarbon chains having 7 carbon atoms, unsaturated hydrocarbon chains having 7 carbon atoms, and mixtures thereof.

Preferred hydroxamic acids are selected from caprylhydroxamic acid, hexanohydroxamic acid, caprohydroxamic acid, laurohydroxamic acid, and mixtures thereof. A particularly preferred hydroxamic acid is caprylhydroxamic acid. Caprylhydroxamic acid may also be referred to as caprylohydroxamic acid or octanohydroxamic acid. Hydroxamic acids are described in, e.g., EP2224973.

Examples of salts of hydroxamic acids are alkali metal salts of hydroxamic acids (e.g. sodium salts of hydroxamic acids or potassium salts of hydroxamic acids) or alkaline earth metal salts of hydroxamic acids (e.g. magnesium salts of hydroxamic acids or calcium salts of hydroxamic acids).

In at least one embodiment, the compound according to Formula (P) is selected from the group consisting of 2-hydroxypyridine-N-oxide, 2-pyridinethiol-1-oxide and salts thereof, 1-hydroxy-4-methyl-6-(2,4,4-trimethylpentyl)-2(1H)-pyridone and salts thereof (preferably the monoethanolamine salt), and mixtures thereof. Formula (P) discloses and encompasses the tautomeric equivalents of these compounds, since an equilibrium always exists. In at least one embodiment, the compound according to Formula (P) is piroctone olamine (Octopirox).

In at least one embodiment, the alkyl diols are selected from the group consisting of 1,2-pentanediol, 1,2-hexanediol, 1,6-hexanediol, 1,2-octanediol, 1,2-heptanediol, 1,2-decanediol, methylpropanediol, and mixtures thereof.

In at least one embodiment, the halogenated compounds are selected from the group consisting of chlorhexidine and salts thereof, triclosan, chlorphenesin, trichlorcarban, chloroxylenol, iodoproprinyl butylcarbamate, bronopol, climbazole, and mixtures thereof.

In at least one embodiment, the isothiazolinones are selected from the group consisting of methylisothiazolinone, methylchloroisothiazolinone, benzylisothiazolinone, and mixtures thereof.

In at least one embodiment, the antimicrobial agent is selected from the group consisting of phenoxyethanol, benzyl alcohol, phenethyl alcohol, benzoic acid and salts thereof, caprylhydroxamic acid, piroctone olamine, and mixtures thereof.

In a preferred embodiment, the antimicrobial agent is 1,2-octanediol, in particular bio-based 1,2-octanediol, for example bio-1,2-octanediol as disclosed in WO 2019/152569.

In at least one embodiment, the antimicrobial blend of the present invention comprises a preservative booster.

In at least one embodiment, the preservative booster is selected from the group consisting of compounds of Formula (VIII)

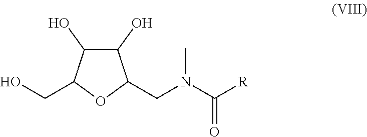

wherein R is selected from saturated hydrocarbon chains having 5 to 23 carbon atoms, unsaturated hydrocarbon chains having 5 to 23 carbon atoms, and mixtures thereof,
sorbitan esters, isosorbide esters, glyceryl ethers, and glyceryl esters.

In at least one embodiment, R in Formula (VIII) is selected from saturated hydrocarbon chains having 5 to 17 carbon atoms, unsaturated hydrocarbon chains having 5 to 17 carbon atoms, and mixtures thereof.

More preferably, R in Formula (VIII) is selected from saturated hydrocarbon chains having 5 to 13 carbon atoms, unsaturated hydrocarbon chains having 5 to 13 carbon atoms, and mixtures thereof.

Particularly preferably, R in Formula (VIII) is selected from saturated hydrocarbon chains having 7 to 9 carbon atoms, unsaturated hydrocarbon chains having 7 to 9 carbon atoms, and mixtures thereof.

In at least one embodiment, R in Formula (VIII) is selected from —(CH₂)₆CH₃, —(CH₂)₈CH₃, and mixtures thereof; or R in Formula (VIII) is selected from —(CH₂)₁₀CH₃, —(CH₂)₁₂CH₃, and mixtures thereof; or R in Formula (VIII) is selected from —(CH₂)₇CH═CH₂, —(CH₂)₇CH═CHCH₂CH₃, and mixtures thereof; or the R—C═O residue in Formula (VIII) is derived from coconut fatty acids; or the R—C═O residue in Formula (VIII) is derived from soybean fatty acids.

In at least one embodiment, R in Formula (VIII) is selected from —(CH₂)₆CH₃, —(CH₂)₈CH₃, and mixtures thereof. In at least one embodiment, R in Formula (VIII) is selected from —(CH₂)₁₀CH₃, —(CH₂)₁₂CH₃, and mixtures thereof. In at least one embodiment, R in Formula (VIII) is selected from —(CH₂)₇CH═CH₂, —(CH₂)₇CH═CHCH₂CH₃, and mixtures thereof. In at least one embodiment, the R—C═O residue in Formula (VIII) is derived from coconut fatty acids. In at least one embodiment, the R—C═O residue in Formula (VIII) is derived from soybean fatty acids.

As used herein, the expression “the R—C═O residue in Formula (VIII) is derived from coconut fatty acids” preferably means that the carbon chain length distribution in the R—C═O residue in Formula (VIII) corresponds to the carbon chain length distribution of the fatty acids (e.g. bound as triglycerides) in coconut oil.

As used herein, the expression “the R—C═O residue in Formula (VIII) is derived from soybean fatty acids” preferably means that the carbon chain length distribution in the R—C═O residue in Formula (VIII) corresponds to the carbon chain length distribution of the fatty acids (e.g. bound as triglycerides) in soybean oil.

Preferably, the weight ratio of compounds according to Formula (VIII) wherein R is —(CH₂)₆CH₃ and compounds according to Formula (VIII) wherein R is —(CH₂)₈CH₃ is from 1:9 to 9:1, preferably from 3:7 to 7:3.

Preferably, the weight ratio of compounds according to Formula (VIII) wherein R is —(CH₂)₁₀CH₃ and compounds according to Formula (VIII) wherein R is —(CH₂)₁₂CH₃ is from 1:9 to 9:1, preferably from 3:7 to 7:3.

Preferably, the weight ratio of compounds according to Formula (VIII) wherein R is —(CH₂)₇CH═CH₂ and compounds according to Formula (VIII) wherein R is —(CH₂)₇CH═CHCH₂CH₃ is from 1:9 to 9:1, preferably from 3:7 to 7:3.

Compounds of Formula (VIII) are described in, e.g., WO 2018/002100 and EP application number EP21200587.0.

In at least one embodiment, the sorbitan esters are mono-, di- or triesters of sorbitan and one or more C6-C20 fatty acids. More preferably, the sorbitan esters are mono- or diesters of sorbitan and one or more C8-C14 fatty acids. Particularly preferably, the sorbitan esters are mono- or diesters of sorbitan and caprylic acid.

Preferably, the sorbitan esters are selected from sorbitan caprylate, sorbitan stearate, sorbitan olivate, sorbitan oleate, sorbitan caprate, sorbitan laurate, sorbitan myristate, sorbitan caproate, and mixtures thereof. Particularly preferably, the sorbitan ester is sorbitan caprylate.

In at least one embodiment, the isosorbide esters are mono- or diesters of isosorbide and one or more C6-C20 fatty acids. More preferably, the isosorbide esters are mono- or diesters of isosorbide and one or more C8-C14 fatty acids. Particularly preferably, the isosorbide esters are mono- or diesters of isosorbide and caprylic acid.

Preferably, the isosorbide esters are selected from isosorbide caprylate, isosorbide stearate, isosorbide olivate, isosorbide oleate, isosorbide caprate, isosorbide laurate, isosorbide myristate, isosorbide caproate, and mixtures thereof. Particularly preferably, the isosorbide ester is isosorbide caprylate.

In at least one embodiment, the glyceryl ethers are mono- or diethers of glycerin and one or more C6-C20 fatty alcohols. More preferably, the glyceryl ethers are mono- or diethers of glycerin and one or more C8-C14 fatty alcohols. Particularly preferably, the glyceryl ethers are monoethers of glycerin and one or more C8 fatty alcohols.

Preferably, the glyceryl ethers are selected from ethylhexylglycerin, methylheptylglycerin, caprylyl glyceryl ether, and mixtures thereof.

In at least one embodiment, the glyceryl ester is glyceryl caprylate and/or glyceryl caprate, for example glyceryl caprylate/caprate.

In at least one embodiment, the blend is an aqueous solution.

In at least one embodiment, the blend of the invention further comprises a solvent. In at least one embodiment, the solvent comprises water and/or alcohol. In at least one embodiment, the solvent is cosmetically acceptable. In a particularly preferred embodiment, the solvent is water. Water is useful for economic reasons but also because it is cosmetically acceptable. Optionally, the blend of the invention comprises water-miscible or water-soluble solvents, such as C1-C5 alkyl monohydric alcohols, preferably C2-C3 alkyl monohydric alcohols.

In a preferred embodiment, the blend of the invention comprises a solvent selected from the group consisting of water, glycols, ethanol, and mixtures thereof. In a particularly preferred embodiment, the blend of the invention comprises water as the solvent.

In a preferred embodiment, the blend of the invention comprises an aqueous, alcoholic or aqueous-alcoholic solvent, wherein the aqueous, alcoholic or aqueous-alcoholic solvent comprises water, ethanol, propanol, isopropanol, 1,2-propylene glycol, 1,3-propylene glycol, isobutanol, butanol, butyl glycol, butyl diglycol, glycerol, or mixtures thereof; preferably wherein the aqueous, alcoholic or aqueous-alcoholic solvent comprises water, ethanol, propanol, isopropanol, 1,2-propylene glycol, 1,3-propylene glycol, glycerol, or mixtures thereof; more preferably wherein the aqueous, alcoholic or aqueous-alcoholic solvent comprises water, isopropanol, 1,2-propylene glycol, 1,3-propylene glycol, or mixtures thereof; even more preferably wherein the aqueous, alcoholic or aqueous-alcoholic solvent consists of water or consists of a mixture of water and an alcohol wherein the alcohol is selected from the group consisting of isopropanol, 1,2-propylene glycol and 1,3-propylene glycol.

The blend of the present invention can be prepared by methods known in the art, e.g. by mixing its ingredients.

The mixture of the invention or the compound of Formula (I) or the antimicrobial blend of the invention can, for example, be used as an antimicrobial agent.

Accordingly, the invention also relates to the use of a mixture of the invention or a compound of Formula (I) or an antimicrobial blend of the invention as an antimicrobial agent.

In a preferred embodiment, the mixture of the invention or the compound of Formula (I) or the antimicrobial blend of the invention is used as an antimicrobial agent in a cosmetic formulation or a household cleaning formulation.

In one more preferred embodiment, the mixture of the invention or the compound of Formula (I) or the antimicrobial blend of the invention is used as an antimicrobial agent in a cosmetic formulation.

In another more preferred embodiment, the mixture of the invention or the compound of Formula (I) or the antimicrobial blend of the invention is used as an antimicrobial agent in a household cleaning formulation.

The mixture of the invention or the compound of Formula (I) or the antimicrobial blend of the invention can, for example, be used as surfactant, emulsifier, and/or solubilizer.

Accordingly, the invention also relates to the use of a mixture of the invention or a compound of Formula (I) or an antimicrobial blend of the invention as surfactant, emulsifier, and/or solubilizer.

In a preferred embodiment, the mixture of the invention or the compound of Formula (I) or the antimicrobial blend of the invention is used as surfactant, emulsifier, and/or solubilizer in a cosmetic formulation or a household cleaning formulation.

In one more preferred embodiment, the mixture of the invention or the compound of Formula (I) or the antimicrobial blend of the invention is used as surfactant, emulsifier, and/or solubilizer in a cosmetic formulation.

In another more preferred embodiment, the mixture of the invention or the compound of Formula (I) or the antimicrobial blend of the invention is used as surfactant, emulsifier, and/or solubilizer in a household cleaning formulation.

The mixture of the invention or the compound of Formula (I) or the antimicrobial blend of the invention can, for example, also be used in oil or mining applications or technical or industrial applications.

The invention also relates to the use of a mixture of the invention or a compound of Formula or an antimicrobial blend of the invention (I) as corrosion inhibitor, gas hydrate inhibitor, wax dispersant, foaming agent, defoaming agent, flotation agent, fuel additive, surfactant or emulsifier, or in fracking fluids, enhanced oil recovery formulations or pipeline cleaning fluids. For example, the mixture of the invention or the compound of Formula (I) or the antimicrobial blend of the invention is used as surfactant for enhanced oil recovery or as surfactant for well treatment. For example, the mixture of the invention or the compound of Formula (I) or the antimicrobial blend of the invention is used as emulsifier for explosives.

The invention also relates to a formulation comprising a mixture of the invention or a compound of Formula (I) or an antimicrobial blend of the invention; and one or more further components.

In one embodiment, the formulation comprises a mixture of the invention and one or more further components. In another embodiment, the formulation comprises a compound of Formula (I) and one or more further components. In another embodiment, the formulation comprises an antimicrobial blend of the invention and one or more further components.

In one embodiment, the formulation comprises from 0.001 to 99.999 wt.-% of the mixture of the invention or the compound of Formula (I) or the antimicrobial blend of the invention, based on the total weight of the formulation.

In one embodiment, the formulation comprises from 0.01 to 99.99 wt.-%, preferably from 0.1 to 99.9 wt.-%, more preferably from 0.1 to 80 wt.-%, even more preferably from 0.1 to 50 wt.-%, particularly preferably from 0.1 to 20 wt.-%, of the mixture of the invention or the compound of Formula (I) or the antimicrobial blend of the invention, based on the total weight of the formulation.

In one embodiment, the formulation comprises from 0.1 to 20 wt.-%, preferably from 0.1 to 10 wt.-%, more preferably from 0.5 to 10 wt.-%, even more preferably from 1 to 10 wt.-%, particularly preferably from 1 to 5 wt.-%, for example 2 to 3 wt.-%, also particularly preferably from 5 to 10 wt.-%, for example 7 to 8 wt.-%, of the mixture of the invention or the compound of Formula (I) or the antimicrobial blend of the invention, based on the total weight of the formulation.

In one embodiment, the formulation comprises from 0.1 to 20 wt.-%, preferably from 1 to 20 wt.-%, more preferably from 5 to 20 wt.-%, even more preferably from 10 to 20 wt.-%, particularly preferably from 12 to 18 wt.-%, for example 15 wt.-%, of the mixture of the invention or the compound of Formula (I) or the antimicrobial blend of the invention, based on the total weight of the formulation.

In one embodiment, the formulation comprises
(a) from 0.01 to 10 wt-%, preferably from 0.1 to 2 wt-%, of a compound of Formula (I) of the present invention or an antimicrobial blend of the present invention; and
(b) from 90 to 99.99 wt-%, preferably from 98 to 99.9 wt-%, of one or more further components.

The present invention also relates to a formulation comprising
(a) from 0.01 to 10 wt-%, preferably from 0.1 to 2 wt-%, of a compound of Formula (I) of the present invention or an antimicrobial blend of the present invention; and
(b) from 90 to 99.99 wt-%, preferably from 98 to 99.9 wt-%, of one or more further components.

In at least one embodiment, the formulation is selected from the group consisting of cosmetic formulations and household cleaning formulations.

In at least one embodiment, the formulation is selected from the group consisting of shampoo, body wash, facial cleanser, face mask, bubble bath, intimate wash, bath oil, cleansing milk, micellar water, make-up remover, cleansing wipes, hair mask, perfume, liquid soap, shaving soap, shaving foam, cleansing foam, day cream, anti-ageing cream, body milk, body lotion, body mousse, face serum, eye cream, sunscreen lotion, sun cream, face cream, after-shave lotion, pre-shaving cream, depilatory cream, skin-whitening gel, self-tanning cream, anti-acne gel, mascara, foundation, primer, concealer, blush, bronzer, blemish balm (bb) cream, eyeliner, night cream, eye brow gel, highlighter, lip stain, hand sanitizer, hair oil, nail varnish remover, conditioner, hair styling gel, hair styling cream, anti-frizz serum, scalp treatment, hair colorant, split end fluid, deodorant, antiperspirant, baby cream, insect repellent, hand cream, sunscreen gel, foot cream, exfoliator, body scrub, cellulite treatment, bar soap, cuticle cream, lip balm, hair treatment, eye shadow, bath additive, body mist, eau de toilette, mouthwash, toothpaste, lubricating gel, moisturizer, serum, toner, aqua sorbet, cream gel, styling mousse, dry shampoo, lip stick, lip gloss, hydro-alcoholic gel, body oil, shower milk, illuminator, lip crayon, hair spray, combing cream, and sunblock.

In at least one embodiment, the formulation is a formulation selected from the group consisting of shampoo, body wash, facial cleanser, cleansing mask, bubble bath, bath oil, cleansing milk, micellar water, make-up remover, cleansing wipes, perfume, soaps, shaving soaps, shaving foams and cleansing foams.

In at least one embodiment, the formulation is a cosmetic formulation for cleansing hair and/or skin.

In at least one embodiment, the formulation is an aqueous solution.

In at least one embodiment, the formulation further comprises a solvent. In at least one embodiment, the solvent comprises water and/or alcohol. In at least one embodiment, the solvent is cosmetically acceptable. In a particularly preferred embodiment, the solvent is water. Water is useful for economic reasons but also because it is cosmetically acceptable. Optionally, the formulation comprises water-miscible or water-soluble solvents, such as C1-C5 alkyl monohydric alcohols, preferably C2-C3 alkyl monohydric alcohols.

In a preferred embodiment, the formulation comprises a solvent selected from the group consisting of water, glycols, ethanol, and mixtures thereof. In a particularly preferred embodiment, the formulation comprises water as the solvent.

In a preferred embodiment, the formulation comprises an aqueous, alcoholic or aqueous-alcoholic solvent, wherein the aqueous, alcoholic or aqueous-alcoholic solvent comprises water, ethanol, propanol, isopropanol, 1,2-propylene glycol, 1,3-propylene glycol, isobutanol, butanol, butyl glycol, butyl diglycol, glycerol, or mixtures thereof; preferably wherein the aqueous, alcoholic or aqueous-alcoholic solvent comprises water, ethanol, propanol, isopropanol, 1,2-propylene glycol, 1,3-propylene glycol, glycerol, or mixtures thereof; more preferably wherein the aqueous, alcoholic or aqueous-alcoholic solvent comprises water, isopropanol, 1,2-propylene glycol, 1,3-propylene glycol, or mixtures thereof; even more preferably wherein the aqueous, alcoholic or aqueous-alcoholic solvent consists of water or consists of a mixture of water and an alcohol wherein the alcohol is selected from the group consisting of isopropanol, 1,2-propylene glycol and 1,3-propylene glycol.

In at least one embodiment, the formulation comprises additives common in cosmetology, pharmacy, and dermatology, which are hereinafter called auxiliaries. In at least one embodiment, the auxiliary is selected from the group consisting of oily substances, emulsifiers, coemulsifiers, cationic polymers, film formers, superfatting agents, stabilizers, active biogenic substances, glycerol, pearlizing agents, dyes and fragrances, solvents, opacifiers, functional acids, and also protein derivatives such as gelatin, collagen hydrolysates, natural or synthetic-based polypeptides, egg yolk, lecithin, lanolin and lanolin derivatives, fatty alcohols, silicones, deodorants, substances with a keratolytic and keratoplastic action, enzymes, and/or carriers/solvents.

In at least one embodiment, the formulation comprises water soluble vitamins and their derivatives, water soluble amino acids and their salts and/or derivatives, viscosity modifiers, dyes, nonvolatile solvents or diluents (water soluble and insoluble), pearlescent aids, thickeners, foam boosters, additional surfactants or nonionic co-surfactants, pediculocides, pH adjusting agents, perfumes, chelants, proteins, skin active agents, sunscreens, UV absorbers, vitamins, niacinamide, caffeine, minoxidil, and combinations thereof. In at least one embodiment, the formulation comprises from 0 wt.-% to 5 wt.-% vitamins and amino acids, by total weight of the formulation. The formulation may also comprise pigment materials such as inorganic, nitroso, monoazo, diazo, carotenoid, triphenyl methane, triaryl methane, xanthene, quinoline, oxazine, azine, anthraquinone, indigoid, thionindigoid, quinacridone, phthalocyanine, botanical, natural colors, including: water soluble components such as those having C.I. Names. The formulation may comprise from 0 wt.-%, preferably from 0.0001 wt.-% to 5 wt.-% pigment materials.

In at least one embodiment, the formulation comprises an oily substance, which is any fatty substance which is liquid at room temperature (25° C.). In at least one embodiment, the formulation comprises oily substance selected from the group consisting of silicone oils, volatile or nonvolatile, linear, branched or cyclic, optionally with organic modification; phenylsilicones; silicone resins and silicone gums; mineral oils such as paraffin oil or vaseline oil; oils of animal origin such as perhydrosqualene, lanolin; oils of plant origin such as liquid triglycerides, e.g., sunflower oil, corn oil, soybean oil, rice oil, jojoba oil, babusscu oil, pumpkin oil, grapeseed oil, sesame oil, walnut oil, apricot oil, macadamia oil, avocado oil, sweet almond oil, lady's-smock oil, castor oil, triglycerides of caprylic/capric acids, olive oil, peanut oil, rapeseed oil, argan oil, abyssinian oil, and coconut oil; synthetic oils such as purcellin oil, isoparaffins, linear and/or branched fatty alcohols and fatty acid esters, preferably guerbet alcohols having 6 to 18, preferably 8 to 10, carbon atoms; esters of linear (C₆-C₁₃) fatty acids with linear (C₆-C₂₀) fatty alcohols; esters of branched (C₆-C₁₃) carboxylic acids with linear (C₆-C₂₀) fatty alcohols, esters of linear (C₆-C₁₈) fatty acids with branched alcohols, especially 2-ethylhexanol; esters of linear and/or branched fatty acids with polyhydric alcohols (such as dimerdiol or trimerdiol, for example) and/or guerbet alcohols; triglycerides based on (C₆-C₁₀) fatty acids; esters such as dioctyl adipate, diisopropyl dimer dilinoleate; propylene glycols/dicaprylate or waxes such as beeswax, paraffin wax or microwaxes, alone or in combination with hydrophilic waxes, such as cetylstearyl alcohol, for example; fluorinated and perfluorinated oils; fluorinated silicone oils; mixtures of the aforementioned compounds.

In at least one embodiment, the formulation comprises a non-ionic coemulsifier. In at least one embodiment, the non-ionic coemulsifier is selected from adducts of from 0 to 30 mol of ethylene oxide and/or from 0 to 5 mol of propylene oxide with linear fatty alcohols having 8 to 22 carbon atoms, with fatty acids having 12 to 22 carbon atoms, with alkylphenols having 8 to 15 carbon atoms in the alkyl group, and with sorbitan or sorbitol esters; (C₁₂-C₁₈) fatty acid monoesters and diesters of adducts of from 0 to 30 mol of ethylene oxide with glycerol; glycerol monoesters and diesters and sorbitan monoesters and diesters of saturated and unsaturated fatty acids having 6 to 22 carbon atoms and, where appropriate, their ethylene oxide adducts; adducts of from 15 to 60 mol of ethylene oxide with castor oil and/or hydrogenated castor oil; polyol esters and especially polyglycerol esters, such as polyglyceryl polyricinoleate and polyglyceryl poly-12-hydroxystearate, for example. Likewise suitable are mixtures of compounds from one or more of these classes of substance. Examples of suitable ionogenic coemulsifiers include anionic emulsifiers, such as mono-, di- or tri-phosphoric esters, but also cationic emulsifiers such as mono-, di-, or tri-alkyl quats and their polymeric derivatives.

In at least one embodiment, the formulation comprises a cationic polymer. Suitable cationic polymers include those known under the INCI designation “Polyquaternium”, especially Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, and also Polyquaternium 37 & mineral oil & PPG trideceth (Salcare SC95), PVP-dimethylaminoethyl methacrylate copolymer, guar-hydroxypropyltriammonium chlorides, and also calcium alginate and ammonium alginate. It is additionally possible to employ cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as amidomethicones, for example; copolymers of adipic acid and dimethylaminohydroxypropyldiethylenetriamine; polyaminopolyamide and cationic chitin derivatives, such as chitosan, for example.

In at least one embodiment, the formulation comprises a superfatting agent. As superfatting agents it is possible to use substances such as, for example, polyethoxylated lanolin derivatives, lecithin derivatives, polyol fatty acid esters, monoglycerides, or fatty acid alkanol amides, the latter serving simultaneously as foam stabilizers. Moisturizers available include for example isopropyl palmitate, glycerol and/or sorbitol.

In at least one embodiment, the formulation comprises a stabilizer. As stabilizer it is possible to use metal salts of fatty acids, such as magnesium, aluminum and/or zinc stearate, for example.

In at least one embodiment, the formulation comprises a care additive. The formulations can be blended with conventional ceramides, pseudoceramides, cholesterol, cholesterol fatty acid esters, fatty acids, triglycerides, cerebrosides, phospholipids, panthenol and similar substances as a care additive.

Functional acids are acidic substances used to impart a clinical functionality to the skin or hair upon application. Suitable functional acids include alpha-hydroxy acids, beta-hydroxy acids, lactic acid, retinoic acid, and similar substances.

In at least one embodiment, the formulation comprises an astringent. In at least one embodiment, the astringent is selected from the group consisting of magnesium oxide, aluminum oxide, titanium dioxide, zirconium dioxide, zinc oxide, oxide hydrates, aluminum oxide hydrate (boehmite) and hydroxide, chlorohydrates of calcium, magnesium, aluminum, titanium, zirconium or zinc. In at least one embodiment, the formulation comprises from 0.001 wt.-% to 10 wt.-%, or from 0.01 wt.-% to 9 wt.-%, or from 0.05 wt.-% to 8 wt.-%, or from 0.1 wt.-% to 5 wt.-% astringent.

In at least one embodiment, the formulation comprises a sun protection agent and/or UV filter. Suitable sun protection agents and UV filters are disclosed in WO-2013/017262A1, from page 32, line 11 to the end of page 33. Suitable sun protection agents and UV filters are also disclosed in WO2018/002100A1, page 38, line 1 to page 39, line 23. In at least one embodiment, the formulation comprises from 0.001 wt.-% to 10 wt.-%, preferably from 0.05 wt.-% to 5 wt.-%, even more preferably from 0.1 wt.-% to 3 wt.-%, most preferably from 0.05 wt.-% to 1 wt.-% sun protection agent and/or UV filter. In at least one embodiment, the formulation comprises a photoprotective substance in an amount of from 0.01 to 10 wt.-%, or from 0.1 to 5 wt.-%, more preferably from 0.2 to 2 wt.-%. Suitable photoprotective substances include, in particular, all of the photoprotective substances specified in EP1084696A1, which is incorporated herein by reference. In a preferred embodiment, the photoprotective substance is selected from the group consisting of 2-ethylhexyl 4-methoxycinnamate, methyl methoxycinnamate, 2-hydroxy-4-methoxybenzophenone-5-sulfonic acid, polyethoxylated p-aminobenzoates, and combinations thereof.

In at least one embodiment, the formulation comprises an anti-oxidant. In at least one embodiment, the anti-oxidant is selected from the group consisting of amino acids, peptides, sugars, imidazoles, carotinoids, carotenes, chlorogenic acid, lipoic acid, thiols, thiol glycosyl esters, thiol N-acetyl esters, thiol methyl esters, thiol ethyl esters, thiol propyl esters, thiol amyl esters, thiol butyl esters, thiol lauryl esters, thiol palmitoyl esters, thiol oleyl esters, thiol linoleyl esters, thiol cholesteryl esters, thiol glyceryl esters, dilaurylthiodipropionate, distearylthiodipropionate, thiodipropionic acid, metal chelators, hydroxy acids, fatty acids, folic acids, vitamin C, tocopherol, vitamin A, stilbenes, derivatives and combinations thereof. In at least one embodiment, the anti-oxidant is selected from the group consisting of glycine, histidine, tyrosine, tryptophan, urocaninic acid, D,L-carnosine, D-carnosine, L-carnosine, beta-carotene, alpha-carotene, lycopene, dihydrolipoic acid, aurothioglucose, propylthiouracil, thioredoxine, glutathione, cysteine, cystine, cystamine, buthioninsulfoximine, homocysteinsulfoximine, buthioninsulfone, penta-, hexa-, heptathioninsulfoximine, hydroxyfatty acids, palmitic acid, phytinic acid, lactoferrin, citric acid, lactic acid, malic acid, humic acid, bile acid, bilirubin, biliverdin, EDTA, EGTA, linoleic acid, linolenic acid, oleic acid, butylhydroxyanisol, trihydroxybutyrophenone, ubichinon, ubichinol, ascorbylpalmitate, Mg-ascorbylphosphate, ascorbylacetate, vitamin E acetate, vitamin A palmitate, carnosine, mannose, ZnO, ZnSO4, selenium methionine, stilbenes, superoxide dismutase, and combinations thereof. In at least one embodiment, the antioxidant is selected from the group consisting of vitamin A, vitamin A derivatives, vitamin E, vitamin E derivatives, and combinations thereof. In at least one embodiment, the formulation comprises from 0.001 wt.-% to 10 wt.-%, preferably from 0.05 wt.-% to 5 wt.-%, particularly preferably from 0.1 wt.-% to 3 wt.-%, also particularly preferably from 0.05 wt.-% to 1 wt.-% antioxidant.

In at least one embodiment, the formulation comprises a dye or pigment. In at least one embodiment, the formulation comprises at least one pigment. Suitable dyes and pigments are disclosed in WO2013/017262A1 in the table spanning pages 36 to 43. Suitable dyes and pigments are also disclosed in WO2018/002100A1, page 41, line 14 to page 42, line 11. These may be colored pigments which impart color effects to the product mass or to hair, or they may be luster effect pigments which impart luster effects to the product mass or to hair. The color or luster effects on hair are preferably temporary, i.e. they last until the next hair wash and can be removed again by washing the hair with customary shampoos. In at least one embodiment, the formulation comprises a total amount of from 0.01 wt.-% to 25 wt.-%, preferably from 5 wt.-% to 15 wt.-% pigment. In at least one embodiment, the particle size of the pigment is from 1 micron to 200 micron, preferably from 3 micron to 150 micron, more preferably from 10 micron to 100 micron.

The pigments are colorants which are virtually insoluble in the application medium, and may be inorganic or organic. Inorganic-organic mixed pigments are also possible. Preference is given to inorganic pigments. The advantage of inorganic pigments is their excellent resistance to light, weather and temperature. The inorganic pigments may be of natural origin. Suitable dyes and pigments are also disclosed in WO2018/002100A1, page 41, line 14 to page 42, line 11.

In at least one embodiment, the formulation comprises from 0.01 wt.-% to 10 wt.-%, preferably from 0.05 wt.-% to 5 wt.-%, of at least one particulate substance. Suitable substances are, for example, substances which are solid at room temperature (25° C.) and are in the form of particles. In at least one embodiment, the particulate substance is selected from the group consisting of silica, silicates, aluminates, clay earths, mica, insoluble salts, in particular insoluble inorganic metal salts, metal oxides, e.g. titanium dioxide, minerals and insoluble polymer particles are suitable. The particles may be present in the formulation in undissolved, preferably stably dispersed form, and, following application to the keratin substrate and evaporation of the solvent, can deposit on the substrate in solid form. A stable dispersion can be achieved by providing the formulation with a yield point which is large enough to prevent the solid particles from sinking. An adequate yield point can be established using suitable gel formers in a suitable amount. In at least one embodiment, the particulate substance is selected from the group consisting of silica (silica gel, silicon dioxide) and metal salts, in particular inorganic metal salts, where silica is particularly preferred. Metal salts are, for example, alkali metal or alkaline earth metal halides, such as sodium chloride or potassium chloride; alkali metal or alkaline earth metal sulfates, such as sodium sulfate or magnesium sulfate.

In at least one embodiment, the formulation comprises a direct dye. Preferred direct dyes are disclosed in WO2018/002100A1, page 43, line 1 to page 44, line 11. In at least one embodiment, the total quantity of direct dyes in the formulation amounts to 0.01 to 15 wt.-%, preferably 0.1 to 10 wt.-%, most preferred 0.5 to 8 wt.-%.

In at least one embodiment, the formulation comprises a conditioning agent. In at least one embodiment, the conditioning agent is a water insoluble, water dispersible, non-volatile, liquid that forms emulsified, liquid particles. In at least one embodiment, the conditioning agent is a silicone (e.g., silicone oil, cationic silicone, silicone gum, high refractive silicone, or silicone resin), an organic conditioning oil (e.g., hydrocarbon oils, polyolefins, or fatty esters), a cationic conditioning surfactant, a high melting point fatty compound, or combinations thereof.

In at least one embodiment, the conditioning agent is a silicone, and the formulation comprises from 0.01% to 10%, or from 0.1% to 5% silicone conditioning agent, by total weight of the formulation. Suitable silicone conditioning agents, and optional suspending agents for the silicone, are described in U.S. Pat. No. 5,104,646. In at least one embodiment, the formulation comprises a silicone gum selected from the group consisting of polydimethylsiloxane, (polydimethylsiloxane) (methylvinylsiloxane) copolymer, poly(dimethylsiloxane) (diphenylsiloxane) (methylvinylsiloxane) copolymer, and mixtures thereof.

In at least one embodiment, the formulation comprises a terminal aminosilicone. “Terminal aminosilicone” as defined herein means silicone comprising one or more amino groups at one or both ends of the silicone backbone. In at least one embodiment, the formulation is substantially free of any silicone compound comprising pendant amino groups. In an embodiment, the formulation is substantially free of any silicone compound other than terminal aminosilicones. In at least one embodiment, the amino group at at least one terminus of the silicone backbone of the terminal aminosilicone is selected from the group consisting of primary amines, secondary amines and tertiary amines. In at least one embodiment, the formulation comprises a terminal aminosilicone conforming to Formula (S):

(R^{F})ₐG₃₋ₐ-Si—(—OSiG₂)ₙ-O-SiG₃₋ₐ(R^{F})ₐ (S)
wherein
G is hydrogen, phenyl, hydroxy, or C₁-C₈ alkyl, preferably methyl;
a is an integer having a value from 1 to 3, preferably 1;
b is 0, 1 or 2, preferably 1;
n is a number from 0 to 1,999;
R^{F} is a monovalent radical conforming to the general formula C_{q}H_{2q}L, wherein q is an integer having a value from 2 to 8 and L is selected from the following groups: —N(R^{T})CH₂—CH₂—N(R^{T})₂; —N(R^{T})₂; —N(R^{T})₃A⁻;
—N(R^{T})CH₂—CH₂—NR^{T}H₂A⁻; wherein R^{T} is hydrogen, phenyl, benzyl, or a saturated hydrocarbon radical, preferably an alkyl radical having from 1 to 20 carbon atoms; A⁻ is a halide ion.

In at least one embodiment, the terminal aminosilicone corresponding to Formula (S) has a=1, q=3, G=methyl, n is from 1000 to 2500, alternatively from 1500 to 1700, and L is —N(CH₃)₂. A suitable terminal aminosilicone corresponding to Formula (S) has a=O, G=methyl, n is from 100 to 1500, or from 200 to 800, and L is selected from the following groups: —N(R^{T})CH₂—CH₂—N(R^{T})₂; —N(R^{T})₂;
—N(R^{T})₃A⁻; —N(R^{T})CH₂—CH₂—NR^{T}H₂A⁻; wherein R^{T} is hydrogen, phenyl, benzyl, or a saturated hydrocarbon radical, preferably an alkyl radical from having from 1 to 20 carbon atoms; A⁻ is a halide ion, alternatively L is —NH₂. In at least one embodiment, the terminal aminosilicone is selected from the group consisting of bis-aminomethyl dimethicone, bis-aminoethyl dimethicone, bis-aminopropyl dimethicone, bis-aminobutyl dimethicone, and mixtures thereof. In an embodiment, the viscosity of the terminal aminosilicone is from 1,000 to 30,000 cPs, or from 5,000 to 20,000 cPs, measured at 25° C. In at least one embodiment, the formulation comprises from 0.1% to 20%, or from 0.5% to 10%, or from 1% to 6% terminal aminosilicone, by total weight of the formulation.

In at least one embodiment, the formulation comprises a high melting point fatty compound. The high melting point fatty compound has a melting point of 25° C. or higher. In at least one embodiment, the high melting point fatty compound is selected from the group consisting of a fatty alcohol, fatty acid, fatty alcohol derivative, fatty acid derivative, and mixtures thereof. Non-limiting examples of the high melting point compounds are found in International Cosmetic Ingredient Dictionary, Fifth Edition, 1993, and CTFA Cosmetic Ingredient Handbook, Second Edition, 1992. The formulation may comprise from 0.1% to 40%, or from 1% to 30%, or from 1.5% to 16%, or from 1.5% to 8% of a high melting point fatty compound, by total weight of the formulation. This is advantageous in view of providing improved conditioning benefits such as slippery feel during the application to wet hair, softness and moisturized feel on dry hair. In at least one embodiment, the fatty alcohol is selected from the group consisting of: cetyl alcohol, stearyl alcohol, behenyl alcohol, and mixtures thereof. In at least one embodiment, the formulation comprises a linear fatty alcohol, wherein the linear fatty alcohol is comprised in a lamellar gel matrix. A lamellar gel matrix is suitable for providing various conditioning benefits such as slippery feel during the application to wet hair and softness and moisturized feel on dry hair. The linear fatty alcohol may comprise from 8 to 24 carbon atoms. In an embodiment, the linear fatty alcohol is selected from the group consisting of: cetyl alcohol, stearyl alcohol, and mixtures thereof. In an embodiment, the weight ratio of total linear fatty alcohol to terminal aminosilicone is from 0.5:1 to 10:1, or from 1:1 to 5:1, or from 2.4:1 to 2.7:1.

In at least one embodiment, the lamellar gel matrix comprises a cationic conditioning surfactant and a high melting point fatty compound. In view of providing the lamellar gel matrix, the cationic conditioning surfactant and the high melting point fatty compound are contained at a level such that the weight ratio of the cationic surfactant to the high melting point fatty compound is in the range of from 1:1 to 1:10, or from 1:1 to 1:6.

In at least one embodiment, the formulation comprises a cationic conditioning surfactant. In at least one embodiment, the formulation comprises from 0.05% to 3.0%, or from 0.075% to 2.0%, or from 0.1% to 1.0%, of cationic conditioning surfactant by total weight of the formulation. In at least one embodiment, the cationic conditioning surfactant is comprised in a lamellar gel matrix. In other words, the formulation comprises a lamellar gel matrix and the lamellar gel matrix comprises the cationic conditioning surfactant. In an embodiment, cationic conditioning surfactant is according to Formula (C):

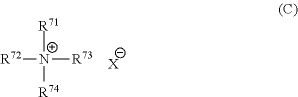

wherein at least one of R⁷¹, R⁷², R⁷³ and R⁷⁴ is selected from an aliphatic group of from 8 to 30 carbon atoms, an aromatic, alkoxy, polyoxyalkylene, alkylamido, hydroxyalkyl, aryl, or an alkylaryl group having up to 22 carbon atoms; the remainder of R⁷¹, R⁷², R⁷³ and R⁷⁴ are independently selected from the group consisting of an aliphatic group of from 1 to 22 carbon atoms, and an aromatic, alkoxy, polyoxyalkylene, alkylamido, hydroxyalkyl, aryl or alkylaryl group having up to 22 carbon atoms;
X is selected from the group consisting of: halogen, acetate, citrate, lactate, glycolate, phosphate, nitrate, sulfonate, sulfate, alkylsulfate, alkyl sulfonate, and combinations thereof.

In at least one embodiment, the cationic conditioning surfactant is selected from the group consisting of behenyl trimethyl ammonium chloride, methyl sulfate or ethyl sulfate, and stearyl trimethyl ammonium chloride, methyl sulfate or ethyl sulfate. It is believed that a longer alkyl group provides improved smoothness and soft feeling on wet and dry hair, compared to cationic surfactants with a shorter alkyl group. It is also believed that such cationic surfactants can provide reduced irritation, compared to those having a shorter alkyl group.

In at least one embodiment, the cationic surfactant is a di-long alkyl quaternized ammonium salt selected from the group consisting of: dialkyl (C14-C18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, and mixtures thereof.

In at least one embodiment, the cationic surfactant is a tertiary amido amine having an alkyl group of from 12 to 22 carbons. The tertiary amido amine may be selected from the group consisting of stearamidopropyldimethyl-, stearamidopropyldiethyl-, stearamidoethyldiethyl-, stearamidoethyldimethyl-, palmitamidopropyldimethyl-, palmitamidopropyldiethyl-, palmitamidoethyldiethyl-, palmitamidoethyldimethyl-, behenamidopropyldimethyl-, behenamidopropyldiethyl-, behenamidoethyldiethyl-, behenamidoethyldimethyl-, arachidamidopropyldimethyl-, arachidamidopropyldiethyl-, arachidamidoethyldiethyl-, and arachidamidoethyldimethyl-amine, diethylaminoethylstearamide, and mixtures thereof. A tertiary amido amine may be used in combination with an acid. The acid is typically used as a salt-forming anion. In an embodiment, the acid is selected from the group consisting of lactic acid, malic acid, hydrochloric acid, 1-glumatic acid, acetic acid, citric acid, and mixtures thereof.

In at least one embodiment, the cationic surfactant is selected from the group consisting of cetyltrimonium chloride (CTAC), stearyltrimonium chloride (STAC), behentrimonium methosulfate, stearoylamidopropyldimethyl amine (SAPDMA), distearyldimethylammonium chloride, and mixtures thereof.

In at least one embodiment, the formulation comprises a surfactant system. In at least one embodiment, the surfactant system comprises a surfactant selected from the group consisting of anionic surfactants, cationic surfactants, non-ionic surfactants, zwitterionic surfactants and/or amphoteric surfactants. In at least one embodiment, the formulation comprises a total amount of surfactant of from 0.01 wt.-% to 70 wt.-%, from 0.1 wt.-% to 40%, from 1 wt.-% to 30%, from 2 wt.-% to 20 wt.-%.

In at least one embodiment, the formulation comprises an anionic surfactant. In at least one embodiment, the anionic surfactant is selected from the group consisting of (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, alpha-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, acylglutamates, and mixtures thereof. The anionic surfactants (and their mixtures) can be used in the form of their water-soluble or water-dispersible salts, examples being the sodium, potassium, magnesium, ammonium, mono, di-, and triethanolammonium, and analogous alkylammonium salts. In at least one embodiment, the anionic surfactant is the salt of an anionic surfactant comprising 12 to 14 carbon atoms. In at least one embodiment, the anionic surfactant is selected from the group consisting of sodium lauryl sulfate, sodium laureth sulfate, sodium tridecyl sulfate, sodium trideceth sulfate, sodium myristyl sulfate, sodium myreth sulfate, and mixtures thereof.

In at least one embodiment, the formulation comprises an acylglycinate surfactant. In at least one embodiment, the acylglycinate surfactant conforms to the formula (Y):

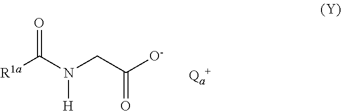

wherein
R^{1a} is a linear or branched, saturated alkyl group having 6 to 30, preferably 8 to 22, particularly preferably 8 to 18 carbon atoms or is a linear or branched, mono- or polyunsaturated alkenyl group having 6 to 30, preferably 8 to 22, particularly preferably 12 to 18 carbon atoms, and
Qₐ⁺ is a cation.

In at least one embodiment, Qₐ⁺ is selected from the group consisting of Li⁺, Na⁺, K⁺, Mg⁺⁺, Ca⁺⁺, Al⁺⁺⁺, NH4⁺, a monoalkylammmonium ion, a dialkylammonium ion, a trialkylammonium ion and a tetraalkylammonium ion, or combinations thereof. In at least one embodiment, the acylglycinate surfactant is selected from sodium cocoylglycinate and potassium cocoylglycinate. In at least one embodiment, the acylglycinate surfactant is selected from those conforming to formula (Y), wherein R is C₁₂ alkyl or C₁₄ alkyl. In at least one embodiment, the acylglycinate surfactant is selected from those conforming to formula (Y), wherein R is C₁₆ alkyl or C₁₈ alkyl.

In at least one embodiment, the formulation comprises a glutamate surfactant corresponding to formula (Z) or a salt thereof:

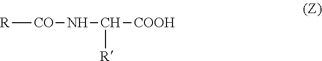

wherein
R′ is HOOC—CH₂—CH₂— or M⁺—OOC—CH₂—CH₂— wherein M⁺ is a cation;
R is a linear or branched, saturated alkyl group having 6 to 30, preferably 8 to 22, more preferably 8 to 18 carbon atoms or is a linear or branched, mono- or polyunsaturated alkenyl group having 6 to 30, preferably 8 to 22, more preferably 12 to 18 carbon atoms. In at least one embodiment, M⁺ is a metal cation. In at least one embodiment, M⁺ is selected from the group consisting of Li⁺, Na⁺, K⁺, Mg⁺⁺, Ca⁺⁺, Al⁺⁺⁺, NH4⁺, a monoalkylammmonium ion, a dialkylammonium ion, a trialkylammonium ion and a tetraalkylammonium ion, or combinations thereof.

In at least one embodiment, the glutamate surfactant is selected from sodium cocoyl glutamate and potassium cocoyl glutamate. In at least one embodiment, the glutamate surfactant is selected from those conforming to formula (Z), wherein R is C₁₂ alkyl or C₁₄ alkyl. In at least one embodiment, the glutamate surfactant is selected from those conforming to formula (Z), wherein R is C₁₆ alkyl or C₁₈ alkyl.

In at least one embodiment, the formulation comprises from 0.01 wt.-% to 30 wt.-%, preferably from 1 wt.-% to 25 wt.-%, more preferably from 5 wt.-% to 20 wt.-%, particularly preferably from 12 wt.-% to 18 wt.-% anionic surfactant.

In at least one embodiment, the formulation comprises a non-ionic surfactant. In at least one embodiment, the non-ionic surfactant has an HLB (Hydrophilic Lipophilic Balance) of greater than 12. Optionally, the non-ionic surfactant is selected from the group consisting of ethoxylated or ethoxylated/propoxylated fatty alcohols with a fatty chain having 12 to 22 carbon atoms, ethoxylated sterols, such as stearyl- or lauryl alcohol (EO-7), PEG-16 soya sterol or PEG-10 soya sterol, polyoxyethylene polyoxypropylene block polymers (poloxamers), and mixtures thereof.

In at least one embodiment, the non-ionic surfactant is selected from the group consisting of ethoxylated fatty alcohols, fatty acids, fatty acid glycerides or alkylphenols, in particular addition products of from 2 to 30 mol of ethylene oxide and/or 1 to 5 mol of propylene oxide onto C8- to C₂₂-fatty alcohols, onto C₁₂- to C₂₂-fatty acids or onto alkyl phenols having 8 to 15 carbon atoms in the alkyl group, C₁₂- to C₂₂-fatty acid mono- and diesters of addition products of from 1 to 30 mol of ethylene oxide onto glycerol, addition products of from 5 to 60 mol of ethylene oxide onto castor oil or onto hydrogenated castor oil, fatty acid sugar esters, in particular esters of sucrose and one or two C₆- to C₂₂-fatty acids, INCI: Sucrose Cocoate, Sucrose Dilaurate, Sucrose Distearate, Sucrose Laurate, Sucrose Myristate, Sucrose Oleate, Sucrose Palmitate, Sucrose Ricinoleate, Sucrose Stearate, esters of sorbitan and one, two or three C₆- to C₂₂-fatty acids and a degree of ethoxylation of from 4 to 20, polyglyceryl fatty acid esters, in particular of one, two or more C₆- to C₂₂-fatty acids and polyglycerol having preferably 2 to 20 glyceryl units, alkyl glucosides, alkyl oligoglucosides and alkyl polyglucosides having C8 to C₂₂-alkyl groups, e.g. decylglucoside or laurylglucoside, and mixtures thereof.

In at least one embodiment, the non-ionic surfactant is selected from the group consisting of fatty alcohol ethoxylates (alkylpolyethylene glycols), alkylphenol polyethylene glycols, alkylmercaptan polyethylene glycols, fatty amine ethoxylates (alkylaminopolyethylene glycols), fatty acid ethoxylates (acylpolyethylene glycols), polypropylene glycol ethoxylates (e.g. Pluronics®), fatty acid alkylol amides (fatty acid amide polyethylene glycols), sucrose esters, sorbitol esters, polyglycol ethers, and mixtures thereof.

In at least one embodiment, the formulation comprises from 1 wt.-% to 20 wt.-%, preferably from 2 wt.-% to 10 wt.-%, more preferably from 3 wt.-% to 7 wt.-% non-ionic surfactant.

In at least one embodiment, the amphoteric surfactants are selected from the group consisting of N—(C₁₂-C₁₈)-alkyl-beta-aminopropionates and N—(C₁₂-C₁₈)-alkyl-beta-iminodipropionates as alkali metal salts and mono-, di-, and trialkylammonium salts; N-acylaminoalkyl-N,N-dimethylacetobetaine, preferably N—(C₈-C₁₈)-acylaminopropyl-N,N-dimethylacetobetaine, (C₁₂-C₁₈)-alkyl-dimethyl-sulfopropylbetaine, amphosurfactants based on imidazoline (e.g. Miranol®, Steinapon®), preferably the sodium salt of 1-(beta-carboxymethyloxyethyl)-1-(carboxymethyl)-2-laurylimidazolinium; amine oxides, e.g. (C₁₂-C₁₈)-alkyldimethylamine oxides, fatty acid amidoalkyldimethylamine oxides, and mixtures thereof.

In at least one embodiment, the formulation comprises a betaine surfactant. Optionally, the betaine surfactant is selected from C₆- to C₁₈-alkylbetaines. In at least one embodiment, the betaine surfactant is selected from the group consisting of cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine and laurylbis(2-hydroxypropyl)alphacarboxyethylbetaine, and combinations thereof. Optionally, the betaine surfactant is selected from C₈- to C₁₈-sulfobetaines. In at least one embodiment, the betaine surfactant is selected from the group consisting of cocodimethylsulfopropylbetaine, stearyldimethylsulfopropylbetaine, lauryldimethylsulfoethylbetaine, laurylbis(2-hydroxyethyl)sulfopropylbetaine, and combinations thereof. Optionally, the betaine surfactant is selected from carboxyl derivatives of imidazole, the C₈- to C₁₈-alkyldimethylammonium acetates, the C₈- to C₁₈-alkyldimethylcarbonylmethylammonium salts, and the C₈- to C₁₈-fatty acid alkylamidobetaines, and mixtures thereof. Optionally, the C₈- to C₁₈-fatty acid alkylamidobetaine is selected from coconut fatty acid amidopropylbetaine, N-coconut fatty acid amidoethyl-N-[2-(carboxymethoxy)ethyl]glycerol (CTFA name: Cocoamphocarboxyglycinate), and mixtures thereof.

In at least one embodiment, the formulation comprises from 0.5 wt.-% to 20 wt.-%, preferably from 1 wt.-% to 10 wt.-% amphoteric surfactant.

In at least one embodiment, the formulation comprises a surfactant system. In at least one embodiment, the surfactant system comprises at least one surfactant selected from the group consisting of lauryl sulfate, laureth sulfate, cocoamido-propylbetaine, sodium cocoylglutamate, lauroamphoacetate, and mixtures thereof. In at least one embodiment, the surfactant system comprises sodium laureth sulphate, sodium lauryl sulphate, and optionally cocamidopropyl betaine. In at least one embodiment, the surfactant system comprises sodium laureth sulphate, potassium cocoyl glutamate, and cocamidopropyl betaine.

In at least one embodiment, the formulation has a viscosity of from 0 cPs to 20,000 cPs. In at least one embodiment, the formulation has a viscosity of from 0.1 cPs to 10,000 cPs, or from 1 cPs to 5,000 cPs, or from 5 cPs to 3,500 cPs.

Viscosity may be important for anti-drip reasons. Dripping can be inconvenient for the user. Furthermore, more viscous formulations can be useful for measured dispensing. In at least one embodiment, the formulation has a viscosity of from 0 cPs to 1,000 cPs. This viscosity range is advantageous when the formulation is in the form of a facial cleanser in view of the need for distribution on skin and ability to rinse off.

In at least one embodiment, the formulation further comprises a viscosity-modifying substance. The viscosity-modifying substance is preferably a thickening polymer.

In at least one embodiment, the thickening polymer is a polymer based on acrylamidomethylpropanesulfonic acid (AMPS®). These polymers, even at pH values of 7 or less, exhibit good thickening performance. Especially preferably, the thickening polymer is selected from the group consisting of homo- or copolymers of acrylamidomethylpropanesulfonic acid and salts thereof. Among the polymers just mentioned, preference is given to polymers having at least 20 mol-% of units based on acrylamidomethylpropanesulfonic acid and/or salts thereof, and particular preference to polymers having at least 50 mol-% of units based on acrylamidomethylpropanesulfonic acid and/or salts thereof, the mole figures relating in each case to the overall polymer. In the case of the copolymers, in addition to structural units based on acrylamidomethylpropanesulfonic acid and/or salts thereof, preferably one or more structural units based on the following comonomers are present in the copolymers: acrylic acid, methacrylic acid, acrylamide, dimethylacrylamide, vinylpyrrolidone (VP), hydroxyethyl acrylate, hydroxyethyl methacrylate, acrylic or methacrylic esters of ethoxylated alcohols RO—(CH₂CH₂O)ₘH, in which R is an alkyl radical having 12 to 30 carbon atoms and m is a number from 3 to 30, and CH₂═CH—COO—(CH₂CH₂—COO)ₙX, in which n is a number from 0 to 10 and X is a counterion and is preferably H⁺, Na⁺ and/or NH4⁺. The polymers selected from the group consisting of homo- or copolymers of acrylamidomethylpropanesulfonic acid and salts thereof may be crosslinked or non-crosslinked. In the case of crosslinking, they contain structural units based on monomers having 2 or more olefinic double bonds. In the case of crosslinking, preferably from 0.1 to 10 mol-% of such structural units are present in the homo- or copolymers, based on the overall polymer. If one or more structural units based on acrylamidomethylpropanesulfonic acid and/or salts thereof in the homo- or copolymers of acrylamidomethylpropanesulfonic acid and/or salts thereof have one or more counterions other than H⁺, these other counterions are preferably selected from the group consisting of Na⁺ and NH4⁺. Suitable polymers are mentioned in publications including EP-0816403, EP-1069142, EP-1116733 and DE-10 2009 014877 (Clariant), EP-1347736 (L'Oréal) or EP-1496081 (Seppic). Examples include: Aristoflex® AVC (Ammonium Acryloyldimethyltaurate/VP Copolymer), Aristoflex® AVS (Sodium Acryloyldimethyltaurate/VP Crosspolymer), Aristoflex® TAC (Ammonium Acryloyl Dimethyltaurate Carboxyethyl Acrylate Crosspolymer), Hostacerin® AMPS (Ammonium Polyacryloyldimethyl Taurate), Aristoflex® HMB (Ammonium Acryloyldimethyltaurate/Beheneth-25 Methacrylate Crosspolymer), Aristoflex® BLV (Ammonium Acryloyldimethyltaurate/Beheneth-25 Methacrylate Crosspolymer), Aristoflex® HMS (Ammonium Acryloyldimethyltaurate/Steareth-25 Methacrylate Crosspolymer), Aristoflex® SNC (Ammonium Acryloyldimethyltaurate/Steareth-8 Methacrylate Copolymer), Aristoflex® LNC (Ammonium Acryloyldimethyltaurate/Laureth-7 Methacrylate Copolymer) or Sepinov® EMT 10 (Hydroxyethyl Acrylate/Sodium Acryloyldimethyl Taurate Copolymer), Sepigel® 305.

In at least one embodiment, the thickening polymer is selected from the group consisting of: copolymers of at least one first monomer type, which is chosen from acrylic acid and methacrylic acid, and at least one second monomer type, which is chosen from esters of acrylic acid and ethoxylated fatty alcohol; crosslinked polyacrylic acid; crosslinked copolymers of at least one first monomer type, which is chosen from acrylic acid and methacrylic acid, and at least one second monomer type, which is chosen from esters of acrylic acid with C₁₀- to C₃₀-alcohols; copolymers of at least one first monomer type, which is chosen from acrylic acid and methacrylic acid, and at least one second monomer type, which is chosen from esters of itaconic acid and ethoxylated fatty alcohol; copolymers of at least one first monomer type, which is chosen from acrylic acid and methacrylic acid, at least one second monomer type, which is chosen from esters of itaconic acid and ethoxylated C₁₀- to C₃₀-alcohols, and a third monomer type, which is chosen from C₁- to C₄-aminoalkyl acrylates; copolymers of two or more monomers chosen from acrylic acid, methacrylic acid, acrylic esters and methacrylic esters; copolymers of vinylpyrrolidone and ammonium acryloyldimethyltaurate; copolymers of ammonium acryloyldimethyltaurate and monomers chosen from esters of methacrylic acid and ethoxylated fatty alcohols; hydroxyethylcellulose; hydroxypropylcellulose; hydroxypropylguar; glyceryl polyacrylate; glyceryl polymethacrylate; copolymers of at least one C₂-, C₃- or C₄-alkylene and styrene; polyurethanes; hydroxypropyl starch phosphate; polyacrylamide; copolymers of maleic anhydride and methyl vinyl ether crosslinked with decadiene; carob seed flour; guar gum; xanthan; dehydroxanthan; carrageenan; karaya gum; hydrolyzed corn starch; copolymers of polyethylene oxide, fatty alcohols and saturated methylenediphenyl diisocyanate (e.g. PEG-150/stearyl alcohol/SMDI copolymer); and mixtures thereof.

In at least one embodiment, the formulation has a pH value of from 2.0 to 12.0, preferably from 3.0 to 9.0, more preferably from 4.5 to 7.5. By varying the pH value, a formulation can be made available that is suitable for different applications.

In at least one embodiment, the formulation comprises an alkalizing agent or pH adjusting agent. In at least one embodiment, ammonia or caustic soda is suitable, but water-soluble, physiologically tolerable salts of organic or inorganic bases can also be considered. Optionally, the pH adjusting agent is selected from ammonium hydrogen carbonate, ammonia, monoethanolamine, ammonium carbonate. In at least one embodiment, the alkalizing agent or pH adjusting agent is selected from the group consisting of 2-amino-2-methyl-1-propanol, 2-amino-2-methyl-1,3-propanediol, 2-amino-2-ethyl-1,3-propanediol, tris(hydroxymethyl)-aminomethane, 2-amino-1-butanol, tris(2-hydroxypropyl)-amine, 2,2-iminobisethanol, lysine, iminourea (guanidine carbonate), tetrahydro-1,4-oxazine, 2-amino-5-guanidin-valeric acid, 2-aminoethansulfonic acid, diethanolamine, triethanolamine, N-methyl ethanolamine, isopropanolamine, diisopropanolamine, triisopropanolamine, glucamine, sodium hydroxide, potassium hydroxide, lithium hydroxide, magnesium oxide, and mixtures thereof.

To establish an acidic pH value, an acid can be included. In at least one embodiment, the formulation comprises an acid selected from the group consisting of hydrochloric acid, phosphoric acid, acetic acid, formic acid, sulfuric acid, citric acid, and mixtures thereof. Citric acid is most preferred in that it has high consumer acceptance. In at least one embodiment, the acidic pH is adjusted with a buffer such as a phosphate buffer, a TRIS buffer or a citric buffer. The buffers may be used alone or in combination with an acid.

In at least one embodiment, the formulation is in liquid form. In an alternative embodiment, the formulation is in solid form. Optionally, the formulation is in powdered or granulated form. This is advantageous in that it is not needed to ship liquid, which is typically heavy, over long distances, which has economic and environmental benefits. A solid form can be achieved by spray drying the formulation or by using a rotary evaporator. The formulation can be converted into liquid form after it has been shipped, e.g. by adding water.

In at least one embodiment, the formulation is a household cleansing formulation.

In at least one embodiment, the formulation is a hand dishwashing formulation. In at least one embodiment, the hand dishwashing formulation comprises an anionic surfactant. In at least one embodiment, the hand dishwashing formulation comprises from 5 wt.-% to 25 wt.-% anionic surfactant. In at least one embodiment, the hand dishwashing formulation comprises a surfactant system comprising at least one anionic surfactant and a further surfactant selected from non-ionic surfactants, amphoteric surfactants, zwitterionic surfactants, and combinations thereof.

Preferably, the hand dishwashing formulation comprises cocoamidopropylbetaine or an amine oxide. Preferably, the amine oxide is lauryl amine oxide, cocoyl amine oxide, or a combination thereof. In at least one embodiment, the pH value of the hand dishwashing formulation is between pH 5.0 and pH 10, preferably between pH 5.5 and pH 9.0. In the case of the hand dishwashing formulation comprising an amine oxide, the hand dishwashing formulation preferably has a pH of between pH 7.5 and pH 9.5, most preferably between pH 8.0 and pH 9.0.

In at least one embodiment, the formulation is a hard surface cleaner. In at least one embodiment, the hard surface cleaner comprises an anionic surfactant. In at least one embodiment, the hard surface cleaner comprises from 1 wt.-% to 10 wt.-% anionic surfactant. In at least one embodiment, the hard surface cleaner comprises a nonionic surfactant. In at least one embodiment, the hard surface cleaner comprises from 1 wt.-% to 10 wt.-% nonionic surfactant. In at least one embodiment, the hard surface cleaner comprises a surfactant system comprising at least one anionic surfactant and a further surfactant selected from non-ionic surfactants, amphoteric surfactants, zwitterionic surfactants, and combinations thereof. Preferably, the hard surface cleaner comprises linear alkylbenzene sulfonate and fatty alcohol ethoxylate. In at least one embodiment, the pH value of the hard surface cleaner is between pH 5.0 and pH 11, preferably between pH 6.0 and pH 9.0.

In at least one embodiment, the formulation is a liquid laundry detergent formulation comprising one or more surfactants. Preferably, the one or more surfactants of the liquid laundry detergent formulation are selected from the group consisting of anionic, nonionic, cationic and zwitterionic surfactants, and more preferably from the group consisting of anionic, nonionic and zwitterionic surfactants.

### Anionic Surfactants

In at least one embodiment, the formulation comprises an anionic surfactant. Anionic surfactants are particularly useful in cleansing formulations such as household cleansing formulations. Preferred anionic surfactants are alkyl sulfonates and alkyl ether sulfates. Preferred alkyl sulfonates are alkylbenzene sulfonates, particularly linear alkylbenzene sulfonates (LAS) having an alkyl chain length of C₆-C₁₅, preferably C₁₂-C₁₄. Possible counterions for concentrated alkaline liquids are ammonium ions, e.g. those generated by the neutralization of alkylbenzene sulfonic acid with one or more ethanolamines, for example monoethanolamine (MEA) and triethanolamine (TEA), or alternatively, alkali metals, e.g. those arising from the neutralization of alkylbenzene sulfonic acid with alkali hydroxides. Preferred alkyl ether sulfates (AES) are alkyl polyethoxylate sulfate anionic surfactants.

### Nonionic Surfactants

In at least one embodiment, the formulation comprises a nonionic surfactant. Nonionic surfactants include primary and secondary alcohol ethoxylates, especially C₈-C₂₀ aliphatic alcohols ethoxylated with an average of from 1 to 20 moles of ethylene oxide per mole of alcohol, and more especially the C₁₀-C₁₅ primary and secondary aliphatic alcohols ethoxylated with an average of from 1 to 10 moles of ethylene oxide per mole of alcohol. Non-ethoxylated nonionic surfactants include alkyl polyglycosides and glycerol monoethers. Mixtures of nonionic surfactants may also be used.

When included therein, the household cleansing formulation, particularly the liquid laundry detergent formulation, preferably comprises from 0.2 wt.-% to 40 wt.-%, more preferably from 1 wt.-% to 20 wt.-% nonionic surfactant, such as alcohol ethoxylate, nonylphenol ethoxylate, alkylpolyglycoside, alkyldimethylamineoxide, ethoxylated fatty acid monoethanolamide, fatty acid monoethanolamide, or combinations thereof. Nonionic surfactants that may be used include the primary and secondary alcohol ethoxylates, especially the C₈-C₂₀ aliphatic alcohols ethoxylated with an average of from 1 to 35 moles of ethylene oxide per mole of alcohol, and more especially the C₁₀-C₁₅ primary and secondary aliphatic alcohols ethoxylated with an average of from 1 to 10 moles of ethylene oxide per mole of alcohol.

### Zwitterionic Surfactants

In at least one embodiment, the formulation comprises a zwitterionic surfactant. The liquid laundry detergent formulation may comprise a zwitterionic surfactant, e.g. amine oxide or betaine, preferably in an amount of up to 10 wt.-% based on the total weight of the liquid laundry detergent formulation. Betaines may be alkyldimethyl betaines or alkylamido betaines, wherein the alkyl groups have C₁₂-C₁₈ chains.

### Additional Surfactants

In at least one embodiment, the liquid laundry detergent formulation comprises a surfactant selected from the group consisting of anionic surfactants, nonionic surfactants, and mixtures thereof; preferably the surfactant is selected from the group consisting of linear alkyl benzene sulfonates, alkyl ether sulfates, nonionic surfactants, amine oxides and betaines; and more preferably selected from the group consisting of linear alkyl benzene sulfonates, alkyl ether sulfates and nonionic surfactants. Other surfactants than the preferred LAS, AES, and nonionic surfactants may also be added. Although less preferred, alkyl sulfate surfactants may be used, especially the non-ethoxylated C₁₂-C₁₅ primary and secondary alkyl sulfates. Soap may also be used. Levels of soap are preferably lower than 10 wt.-%.

Preferably, the one or more surfactants in the liquid laundry detergent formulations are present in an amount of at least 5 wt.-%, preferably from 5 wt.-% to 65 wt.-%, more preferably from 6 wt.-% to 60 wt.-% and particularly preferably from 7 wt.-% to 55 wt.-%, in each case based on the total weight of the liquid laundry detergent formulation.

### Further Optional Ingredients

The household cleansing formulations may comprise one or more optional ingredients, e.g. they may comprise conventional ingredients commonly used in detergent compositions, especially laundry detergent compositions. Examples of optional ingredients include, but are not limited to builders, bleaching agents, bleach active compounds, bleach activators, bleach catalysts, photobleaches, dye transfer inhibitors, color protection agents, anti-redeposition agents, dispersing agents, fabric softening and antistatic agents, fluorescent whitening agents, enzymes, enzyme stabilizing agents, foam regulators, defoamers, malodor reducers, disinfecting agents, hydrotropes, fibre lubricants, anti-shrinkage agents, buffers, fragrances, processing aids, colorants, dyes, pigments, anti-corrosion agents, fillers, stabilizers or other conventional ingredients for washing or laundry detergent compositions.

### Polymer

For detergency boosting, it may be advantageous to use a polymer in the household cleansing formulations, particularly in the liquid laundry detergent formulations. This polymer is preferably a polyalkoxylated polyethyleneimine (EPEI). Polyethylene imines are materials composed of ethylene imine units
—CH₂CH₂NH— and, where branched, the hydrogen on the nitrogen is replaced by another chain of ethylene imine units. These polyethyleneimines can be prepared, for example, by polymerizing ethyleneimine in the presence of a catalyst such as carbon dioxide, sodium bisulfite, sulfuric acid, hydrogen peroxide, hydrochloric acid, acetic acid, or the like. Specific methods for preparing these polyamine backbones are disclosed in U.S. Pat. Nos. 2,182,306, 3,033,746, 2,208,095, 2,806,839, and 2,553,696.

The household cleansing formulations, particularly the liquid laundry detergent formulations, may comprise other polymeric materials, for example: dye transfer inhibition polymers, anti-redeposition polymers or cotton soil release polymers, especially those based on modified cellulosic materials. Especially, when EPEI is not present, the formulation may further comprise a polymer of polyethylene glycol and vinyl acetate, for example the lightly grafted copolymers described in WO 2007/138054. Such amphiphilic graft polymers based on water soluble polyalkylene oxides as graft base and side chains formed by polymerization of a vinyl ester component have the ability to enable reduction of surfactant levels whilst maintaining high levels of oily soil removal.

### Hydrotropes

In at least one embodiment, the formulation comprises a hydrotrope. Herein “hydrotrope” is a solvent that is neither water nor conventional surfactant, and that aids the solubilisation of surfactants and other components, especially any polymer and/or sequestrant, in the liquid, to render it isotropic. Hydrotropes are particularly useful in household cleansing formulations. Among suitable hydrotropes the following are noteworthy: monopropylene glycol (MPG), glycerol, sodium cumene sulfonate, ethanol, other glycols, e.g. dipropylene glycol, diethers and urea. MPG and glycerol are preferred hydrotropes.

### Enzymes

In at least one embodiment, the formulation, particularly the liquid laundry detergent formulation, comprises an enzyme. In at least one embodiment, the enyzme is selected from the group consisting of protease, mannanase, pectate lyase, cutinase, esterase, lipase, amylase, cellulase, and combinations thereof. Less preferred additional enzymes may be selected from peroxidase and oxidase. The enzymes are preferably present with corresponding enzyme stabilizers. The total enzyme content in the formulation is preferably from 0 wt. % to 5 wt.-%, more preferably from 0.5 wt.-% to 5 wt.-%, even more preferably from 1 wt.-% to 4 wt.-%, by total weight of the formulation.

### Sequestrants

Sequestrants are preferably included in the formulation, particularly in the household cleansing formulations. Preferred sequestrants include organic phosphonates, alkanehydroxy phosphonates and carboxylates, for example available under the DEQUEST trade mark from Thermphos. The preferred sequestrant level is less than 10 wt.-% and preferably less than 5 wt.-% by total weight of the formulation. A particularly preferred sequestrant is HEDP (1-hydroxyethylidene-1,1-diphosphonic acid), for example sold as Dequest 2010. Also suitable is Dequest® 2066 (diethylenetriamine penta(methylene-phosphonic acid) or Heptasodium DTPMP).

### Buffers

In at least one embodiment, the formulation, particularly the liquid laundry detergent formulation, comprises a buffer. In addition to agents optionally included for the generation of anionic surfactants, e.g. from LAS or fatty acids, the presence of buffer is preferred for pH control. Possible buffers are one or more ethanolamines, e.g. monoethanolamine (MEA) or triethanolamine (TEA). They are preferably used in formulation at levels of from 1.0 wt.-% to 15 wt.-%. Other suitable amino alcohol buffer materials may be selected from the group consisting of compounds having a molecular weight above 61 g/mol, which includes MEA. Suitable materials also include, in addition to the already mentioned materials: monoisopropanolamine, diisopropanolamine, triisopropanolamine, monoamino hexanol, 2-[(2-methoxyethyl)methylamino]-ethanol, propanolamine, N-methylethanolamine, diethanolamine, monobutanolamine, isobutanolamine, monopentanolamine, 1-amino-3-(2-methoxyethoxy)-2-propanol, 2-methyl-4-(methylamino)-2-butanol, or mixtures thereof. Potential alternatives to amino ethanol buffers are alkali hydroxides such as sodium hydroxide or potassium hydroxide.

The following examples are intended to further illustrate the invention without limiting the scope thereof.

### EXAMPLES

### Example 1—Synthesis of Cyclic N-Methylglucamides

### Example 1a—Synthesis of Cyclic N-Dodecanoyl/Tetradecanoyl-N-Methylglucamide

0.5 mol of a 60% aqueous N-Methylglucamine (NMG) solution is weighed into the reaction flask. While stirring, this solution is heated up to 135° C. to evaporate water.

The pressure is gradually reduced to 20 mbar. Afterwards the temperature is increased to 160° C. and vacuum is broken with nitrogen.

A mixture of 0.485 mol C12/C14 fatty acid and 0.001 mol phosphonic acid (70%) is slowly added to the hot melt of N-Methylglucamine to avoid foaming and gel phases. With a moderate stream of nitrogen, the reaction mixture is stirred at 160° C. for 4 h. A first amount of water is distilling off. The laboratory trial is then interrupted in order to manage the next reaction step within one working day.

Therefore, the batch is cooled down to ambient temperature and stored under nitrogen. The cooling down phase can be skipped in plant where overnight operation is possible.

Before starting the batch again 0.003 mol of phosphonic acid (70%) is added. The whole apparatus is evacuated 3 times and released to ambient pressure with nitrogen. Then the reaction is heated up to 160° C. Afterwards pressure is reduced gradually to 50 mbar. When 50 mbar are reached, the reaction is carried on at 160° C. until the content of fatty acid is <1% according to the titrated acid number. The resultant product contains 92 wt % of the desired cyclic N-Dodecanoyl/Tetradecanoyl-N-Methylglucamide measured by GC after derivatisation.

### Example 1b—Synthesis of Cyclic N-Hexadecanoyl/Octadecanoyl-N-Methylglucamide

Cyclic N-Hexadecanoyl/Octadecanoyl-N-Methylglucamide is prepared in an analogous manner to cyclic N-Dodecanoyl/Tetradecanoyl-N-Methylglucamide (Example 1a). The resultant product contains 76 area-% of the desired cyclic N-Hexadecanoyl/Octadecanoyl-N-Methylglucamide measured by GC after derivatisation.

### Example 1c—Synthesis of Cyclic N-Oleyl-N-Methylglucamide from High Oleic Sunflower Oil

Cyclic N-Oleyl-N-Methylglucamide is prepared in an analogous manner to cyclic N-Dodecanoyl/Tetradecanoyl-N-Methylglucamide (Example 1a). However, instead of a fatty acid, 0.48 mol high oleic sunflower oil is used. The resultant product contains 74 area-% of the desired cyclic N-Oleyl-N-Methylglucamide measured by GC after derivatisation.

### Example 2—Synthesis of Cyclic N-Octanoyl/Decanoyl-N-Methylxylosamide

0.5 mol of a 60% aqueous N-Methylxylosamine (NMX) solution is weighed into the reaction flask. While stirring, this solution is heated up to 135° C. to evaporate water.

The pressure is gradually reduced to 20 mbar. Afterwards the temperature is increased to 160° C. and vacuum is broken with nitrogen.

A mixture of 0.5 mol C8/C10 fatty acid together with 0.001 mol phosphonic acid (70%) is slowly added to the hot melt of N-Methylxylosamine to avoid foaming and gel phases. With a moderate stream of nitrogen, the reaction mixture is stirred at 160° C. for 4 h. A first amount of water is distilling off.

The laboratory trial is then interrupted in order to manage the next reaction step within one working day. Therefore, the batch is cooled down to ambient temperature and stored under nitrogen. The cooling down phase can be skipped in plant where overnight operation is possible.

Before starting the batch again 0.003 mol of phosphonic acid (70%) is added. The whole apparatus is evacuated 3 times and released to ambient pressure with nitrogen. Then the reaction is heated up to 160° C. Afterwards pressure is reduced gradually to 50 mbar. When 50 mbar are reached, the reaction is carried on at 160° C. until the content of fatty acid is <1% according to the titrated acid number. The resultant product contains 85 area-% of the desired cyclic N-Octanoyl/Decanoyl-N-Methylxylosamide measured by GC after derivatisation.

### Example 3—Synthesis of Cyclic N-Ethylglucamides

### Example 3a—Synthesis of Cyclic N-Octanoyl/Decanoyl-N-Ethylglucamide

0.5 mol of a 40% aqueous N-Ethyl-D-glucamine (NEG) solution is weighed into the reaction flask. While stirring, this solution is heated up to 135° C. to evaporate water.

The pressure is gradually reduced to 20 mbar. Afterwards the temperature is increased to 160° C. and vacuum is broken with nitrogen.

A mixture of 0.485 mol C8/C10 fatty acid together with 0.001 mol phosphonic acid (70%) is slowly added to the hot melt of N-Ethyl-D-glucamine to avoid foaming and gel phases. With a moderate stream of nitrogen, the reaction mixture is stirred at 160° C. for 4 h. A first amount of water is distilling off.

The laboratory trial is then interrupted in order to manage the next reaction step within one working day. Therefore, the batch is cooled down to ambient temperature and stored under nitrogen. The cooling down phase can be skipped in plant where overnight operation is possible.

Before starting the batch again 0.002 mol of phosphonic acid (70%) is added. The whole apparatus is evacuated 3 times and released to ambient pressure with nitrogen. Then the reaction is heated up to 160° C. Afterwards pressure is reduced gradually to 50 mbar. When 50 mbar are reached, the reaction is carried on at 160° C. until the content of fatty acid is <1% according to the titrated acid number. The resultant product contains 89 wt % of the desired cyclic N-Octanoyl/Decanoyl-N-Ethylglucamide measured by GC after derivatisation.

### Example 3b—Synthesis of Cyclic N-Dodecanoyl/Tetradecanoyl-N-Ethylglucamide

Cyclic N-Dodecanoyl/Tetradecanoyl-N-Ethylglucamide is prepared in an analogous manner to cyclic N-Octanoyl/Decanoyl-N-Ethylglucamide (Example 3a). The resultant product contains 81 wt % of the desired cyclic N-Dodecanoyl/Tetradecanoyl-N-Ethylglucamide measured by GC after derivatisation.

### Example 3c—Synthesis of Cyclic N-Hexadecanoyl/Octadecanoyl-N-Ethylglucamide

Cyclic N-Hexadecanoyl/Octadecanoyl-N-Ethylglucamide is prepared in an analogous manner to cyclic N-Octanoyl/Decanoyl-N-Ethylglucamide (Example 3a). The resultant product contains 86 wt % of the desired cyclic N-Hexadecanoyl/Octadecanoyl-N-Ethylglucamide measured by GC after derivatisation.

### Example 4—Synthesis of Linear N-Ethylglucamides

### Example 4a—Synthesis of Linear N-Octanoyl/Decanoyl-N-Ethylglucamide

0.27 mol of a 40% aqueous N-Ethyl-D-glucamine (NEG) solution and 0.02 mol of a 50% aqueous NaOH solution are weighed into a reaction flask. While stirring, this solution is heated up to 135° C. to evaporate water. Then 30 mbar vacuum is applied for 30 minutes at 135° C. Afterwards vacuum is broken with nitrogen.

0.14 mol propylene glycol (PG), heated to 110° C., is quickly added to the NEG melt and the temperature is allowed to drop to 120° C.

0.28 mol of a tempered C8/C10 fatty acid methyl ester (FAME) (105-120° C.) is added. The temperature is allowed to drop to 95° C. and the reaction mixture is refluxed for 30 minutes. Then the temperature is set to 85° C. and the pressure is reduced to 30 mbar and Methanol is distilled off for 90-120 min. Afterwards vacuum is broken to ambient pressure with nitrogen. The resultant product contains 73 wt % of the desired linear N-Octanoyl/Decanoyl N-Ethylglucamide measured by GC after derivatisation.

The linear C12/C14 and C16/C18 N-Ethylglucamides are synthesized analogous to the linear C8/C10 N-Ethylglucamide.

| | Linear N- | | | NEG:NaOH:FAME:PG | |
|---|---|---|---|---|---|
| Example | Ethylglucamide | Amine | FAME | [mol] | Yield |
| 4b | Linear | 40% | C12/C14 | 0.27:0.02:0.28:0.17 | 65 wt % |
| | Dodecanoyl/ | aqueous | | | |
| | Tetradecanoyl | NEG | | | |
| | N-Ethylglucamide | solution | | | |
| 4c | Linear | 40% | C16/C18 | 0.27:0.02:0.28:0.19 | 71 wt % |
| | Hexadecanoyl/ | aqueous | | | |
| | Octadecanoyl | NEG | | | |
| | N-Ethylglucamide | solution | | | |

### Example 5—Synthesis of Linear N-Methylxylosamides

### Example 5a—Synthesis of Linear N-Octanoyl/Decanoyl-N-Methylxylosamide

0.27 mol of a 55% aqueous N-Methylxylamine (NMX) solution and 0.02 mol of a 50% aqueous NaOH solution are weighed into the reaction flask. While stirring, this solution is heated up to 135° C. to evaporate water. Then 30 mbar vacuum is applied for 30 minutes at 135° C. Afterwards vacuum is broken with nitrogen.

0.14 mol propylene glycol (PG), heated to 110° C., is quickly added to the NMX melt and the temperature is allowed to drop to 120° C.

0.28 mol of a tempered C8/C10 fatty acid methyl ester (FAME) (105-120° C.) is added. The temperature is allowed to drop to 95° C. and the reaction mixture is refluxed for 30 minutes. Then the temperature is set to 85° C. and the pressure is reduced to 30 mbar and Methanol is distilled off for 90-120 min.

Afterwards vacuum is broken to ambient pressure with nitrogen.

The linear C12/C14 N-Methylxylosamide is synthesized analogous to the linear C8/C10 N-Methylxylosamide.

The C12/14/16/18, C16/18 and C22 N-Methylxylosam ides are synthesized in a similar manner to the linear C8/C10 N-Methylxylosamide: Additionally, 0.01 mol distilled H₂O is added after the addition of propylene glycol.

| | Linear N- | | | NMX:NaOH:FAME:H₂O:PG |
|---|---|---|---|---|
| Example | Methylxylamide | Amine | FAME | [mol] |
| 5c | Linear Dodecanoyl/ | 71% aqueous | C12/C14/C16/C18 | 0.27:0.02:0.28:0.01:0.18 |
| | Tetradecanoyl/ | NMX solution | | |
| | Hexadecanoyl/ | | | |
| | Octadecanoyl | | | |
| | N-Methylxylosamide | | | |
| 5d | Linear | 71% aqueous | C16/C18 | 0.27:0.02:0.28:0.01:0.38 |
| | Hexadecanoyl/ | NMX solution | | |
| | Octadecanoyl | | | |
| | N-Methylxylosamide | | | |
| 5e | Linear Docosanoyl | 71% aqueous | C22 | 0.27:0.02:0.28:0.01:0.37 |
| | N-Methylxylosamide | NMX solution | | |

### Example 6—Synthesis of Linear N-Methylarabinam ides

### Example 6a—Synthesis of Linear N-Dodecanoyl/Tetradecanoyl-N Methylarabinamide

0.27 mol of a 54% aqueous N-Methyl-L-(+)-arabinamine (NMA) solution and 0.02 mol of a 50% aqueous NaOH solution are weighed into the reaction flask. While stirring, this solution is heated up to 135° C. to evaporate water. Then 30 mbar vacuum is applied for 30 minutes at 135° C. Afterwards vacuum is broken with nitrogen

0.14 mol propylene glycol (PG), heated to 110° C., are quickly added to the NMA melt and the temperature is allowed to drop to 120° C.

After addition of 0.01 mol distilled H₂O, 0.28 mol of a tempered C12/C14 fatty acid methyl ester (FAME) (105-120° C.) is added. The temperature is allowed to drop to 95° C. and the reaction mixture is refluxed for 30 minutes. Then the temperature is set to 85° C. and the pressure is reduced to 30 mbar and Methanol is distilled off for 90-120 min. During the distillation the temperature is slowly increased to 100° C., otherwise the product becomes solid at lower temperatures. Afterwards vacuum is broken to ambient pressure with nitrogen. The resultant product contains 79 wt % of the desired linear N-Dodecanoyl/Tetradecanoyl-N Methylarabinamide measured by GC after derivatisation.

The linear C16/18 N-Methylarabinamide is synthesized analogous to the linear C12/C14 N-Methylarabinamide.

| | Linear N- | | | | | |
|---|---|---|---|---|---|---|
| | Methyl- | | | NMA:NaOH:FAME:H₂O:PG | | |
| Example | arabinamide | Amine | FAME | [mol] | Comments | Yield |
| 6b | Linear | 41% aqueous | C16/C18 | 0.27:0.02:0.28:0.01:0.37 | End | 67 wt % |
| | Hexadecanoyl/ | NMA solution | | | temperature | |
| | Octadecanoyl | from | | | is 95° C. | |
| | N-Methyl- | D-(−)- | | | | |
| | arabinamide | Arabinose | | | | |

### Example 7—Synthesis of Linear N-Cocoyl-N-Butylglucamide

0.27 mol N-Butylglucamine (NBG), 13 mol MeOH, 0.02 mol of a 30% methanolic NaOMe solution and 0.11 mol Coconut Oil are weighed into the reaction flask. The mixture is stirred at 60° C. until complete dissolution. The temperature is set to 90° C., while a first amount of MeOH is distilled off. Then 0.20 mol propylene glycol are added to the reaction mixture and the temperature is set to 85° C. The pressure is reduced to 25 mbar and further MeOH is distilled off for 5 hours. The resultant product contains 75 wt % of the desired linear N-Cocoyl-N-Butylglucamide measured by GC after derivatisation.

### Example 8—Synthesis of Linear N-Octylglucamides

### Example 8a—Synthesis of Linear N-Dodecanoyl/Tetradecanoyl-N-Octylglucamide

0.27 mol N-Octylglucamine (NOG), 18 mol MeOH, 0.02 mol of a 30% methanolic NaOMe solution and 0.26 mol C12/C14 fatty acid methyl ester (FAME) are weighed into the reaction flask. The mixture is stirred at 65° C. until complete dissolution. The temperature is set to 85° C., while a first amount of MeOH is distilled off. Then 0.18 mol propylene glycol (PG) are added to the reaction mixture. The pressure is reduced to 25 mbar and further MeOH is distilled off for 5 hours. The resultant product contains 74 wt % of the desired linear N-Dodecanoyl/Tetradecanoyl-N-Octylglucamide measured by GO after derivatisation.

The linear C16/C18, C12/18 and Cocoyl N-Octylglucamides are synthesized analogous to the linear C12/C14 N-Octylglucamide.

| | Linear N-Octyl- | | | NOG:NaOMe:MeOH:FAME/Oil:PG | |
|---|---|---|---|---|---|
| Example | glucamide | Amine | FAME/Oil | [mol] | Yield |
| 8b | Linear Hexadecanoyl/ | 12% | C16/C18 | 0.27:0.02:18:0.32:0.49 | 58 |
| | Octadecanoyl | methanolic | | | wt % |
| | N-Octylglucamide | NOG solution | | | |
| 8c | Linear Dodecanoyl/ | 12% | C12/C14/C16/C18 | 0.27:0.02:18:0.28:0.25 | 66 |
| | Tetradecanoyl/ | methanolic | | | wt % |
| | Hexadecanoyl/ | NOG solution | | | |
| | Octadecanoyl | | | | |
| | N-Octylglucamide | | | | |
| 8d | Linear Cocoyl | 12% | Coconut Oil | 0.27:0.02:18:0.09:0.21 | 65 |
| | N-Octylglucamide | methanolic | | | wt % |
| | | NOG solution | | | |

### Example 9—Synthesis of Linear N-Methylarabinamide-N-Methylxylamide Mixtures

### Example 9a—Synthesis of Linear Octanoyl/Decanoyl N-Methylarabinamide-N-Methylxylamide Mixture

0.27 mol of a mixture consisting of 87% of a 69% aqueous N-Methylxylamine (NMX) solution and 13% of a 41% aqueous Methylarabinamine (NMA) solution is weighed into the reaction flask. 0.02 mol of a 50% aqueous NaOH solution are added. While stirring, this NMX-NMA-mixture is heated up to 135° C. to evaporate water. Then 30 mbar vacuum is applied for 30 minutes at 135° C. Afterwards vacuum is broken with nitrogen

0.12 mol propylene glycol (PG), heated to 110° C., are quickly added to the NMA/NMX melt and the temperature is allowed to drop to 120° C.

After addition of 0.01 mol distilled water, 0.28 mol of C8/C10 fatty acid methyl ester (FAME) (105-120° C.) is added. The temperature is allowed to drop to 95° C. and the reaction mixture is refluxed for 30 minutes. Then the temperature is set to 85° C. and the pressure is reduced to 30 mbar and Methanol is distilled off for 90-120 min. Afterwards vacuum is broken to ambient pressure with nitrogen. The resultant product contains 70 wt % of the desired linear Octanoyl/Decanoyl N-Methylarabinamide-N-Methylxylamide mixture measured by GC after derivatisation.

### Example 9b—Synthesis of Linear N-Dodecanoyl/Tetradecanoyl N-Methylarabinamide-N-Methylxylamide Mixture

Linear N-Dodecanoyl/Tetradecanoyl N-Methylarabinamide-N-Methylxylamide mixture is prepared in an analogous manner to linear Octanoyl/Decanoyl N-Methylarabinamide-N-Methylxylamide mixture (Example 9a). The resultant product contains 75 wt % of the desired linear N-Dodecanoyl/Tetradecanoyl N-Methylarabinamide-N-Methylxylamide mixture measured by GC after derivatisation.

### Example 10—Example Mixtures of the Present Invention

Mixtures of a compound of Formula (I) and a compound of Formula (II) wherein the weight ratio of the compound of Formula (I) to the compound of Formula (II) is as indicated in Table 1.

TABLE 1

| | Compound of Formula (I) | Compound of Formula (II) wherein | |
|---|---|---|---|
| | wherein R¹ is methyl; and R² is as | R³ is methyl; and R⁴ is as | |
| | indicated in this column | indicated in this column | |
| | 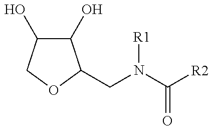 | 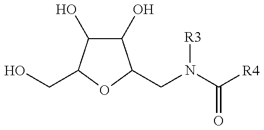 | |
| Example | (derived from xylose) | (derived from glucose) | Weight ratio |
| 10a | C7 alkyl (unbranched) | C7 alkyl (unbranched) | 1:1 |
| 10b | C7 alkyl (unbranched) | C9 alkyl (unbranched) | 1:2 |
| 10c | C7 alkyl (unbranched) | C11 alkyl (unbranched) | 2:1 |
| 10d | C7 alkyl (unbranched) | C13 alkyl (unbranched) | 1:1 |
| 10e | C7 alkyl (unbranched) | C15 alkyl (unbranched) | 1:2 |
| 10f | C7 alkyl (unbranched) | C17 alkyl (unbranched) | 2:1 |
| 10g | C9 alkyl (unbranched) | C7 alkyl (unbranched) | 1:1 |
| 10h | C9 alkyl (unbranched) | C9 alkyl (unbranched) | 1:2 |
| 10i | C9 alkyl (unbranched) | C11 alkyl (unbranched) | 2:1 |
| 10j | C9 alkyl (unbranched) | C13 alkyl (unbranched) | 1:1 |
| 10k | C9 alkyl (unbranched) | C15 alkyl (unbranched) | 1:2 |
| 10l | C9 alkyl (unbranched) | C17 alkyl (unbranched) | 2:1 |
| 10m | C11 alkyl (unbranched) | C7 alkyl (unbranched) | 1:1 |
| 10n | C11 alkyl (unbranched) | C9 alkyl (unbranched) | 1:2 |
| 10o | C11 alkyl (unbranched) | C11 alkyl (unbranched) | 2:1 |
| 10p | C11 alkyl (unbranched) | C13 alkyl (unbranched) | 1:1 |
| 10q | C11 alkyl (unbranched) | C15 alkyl (unbranched) | 1:2 |
| 10r | C11 alkyl (unbranched) | C17 alkyl (unbranched) | 2:1 |
| 10s | C13 alkyl (unbranched) | C7 alkyl (unbranched) | 1:1 |
| 10t | C13 alkyl (unbranched) | C9 alkyl (unbranched) | 1:2 |
| 10u | C13 alkyl (unbranched) | C11 alkyl (unbranched) | 2:1 |
| 10v | C13 alkyl (unbranched) | C13 alkyl (unbranched) | 1:1 |
| 10w | C13 alkyl (unbranched) | C15 alkyl (unbranched) | 1:2 |
| 10x | C13 alkyl (unbranched) | C17 alkyl (unbranched) | 2:1 |
| 10y | C15 alkyl (unbranched) | C7 alkyl (unbranched) | 1:1 |
| 10z | C15 alkyl (unbranched) | C9 alkyl (unbranched) | 1:2 |
| 10aa | C15 alkyl (unbranched) | C11 alkyl (unbranched) | 2:1 |
| 10bb | C15 alkyl (unbranched) | C13 alkyl (unbranched) | 1:1 |
| 10cc | C15 alkyl (unbranched) | C15 alkyl (unbranched) | 1:2 |
| 10dd | C15 alkyl (unbranched) | C17 alkyl (unbranched) | 2:1 |
| 10ee | C17 alkyl (unbranched) | C7 alkyl (unbranched) | 1:1 |
| 10ff | C17 alkyl (unbranched) | C9 alkyl (unbranched) | 1:2 |
| 10gg | C17 alkyl (unbranched) | C11 alkyl (unbranched) | 2:1 |
| 10hh | C17 alkyl (unbranched) | C13 alkyl (unbranched) | 1:1 |
| 10ii | C17 alkyl (unbranched) | C15 alkyl (unbranched) | 1:2 |
| 10jj | C17 alkyl (unbranched) | C17 alkyl (unbranched) | 2:1 |

### Example 11—Example Mixtures of the Present Invention

Mixtures of a compound of Formula (I) and a compound of Formula (II) wherein the weight ratio of the compound of Formula (I) to the compound of Formula (II) is as indicated in Table 2.

TABLE 2

| | Compound of Formula (I) wherein | Compound of Formula (II) wherein | |
|---|---|---|---|
| | R¹ is methyl; and R² is as | R³ is methyl; and R⁴ is as | |
| | indicated in this column | indicated in this column | |
| | 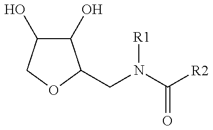 | 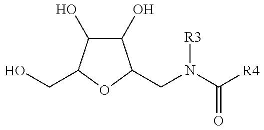 | |
| Example | (derived from xylose) | (derived from glucose) | Weight ratio |
| 11a | C7/C9 alkyl (unbranched) | C7/C9 alkyl (unbranched) | 1:1 |
| 11b | C7/C9 alkyl (unbranched) | C11/C13 alkyl (unbranched) | 1:2 |
| 11c | C7/C9 alkyl (unbranched) | C15/C17 alkyl (unbranched) | 2:1 |
| 11d | C7/C9 alkyl (unbranched) | —(CH₂)₇CH═CH₂ | 1:1 |
| 11e | C7/C9 alkyl (unbranched) | —(CH₂)₇CH═CHCH₂CH₃ | 1:2 |
| 11f | C7/C9 alkyl (unbranched) | the R⁴—C═O residue in Formula (II) is | 2:1 |
| | | cocoyl | |
| 11g | C11/C13 alkyl (unbranched) | C7/C9 alkyl (unbranched) | 1:1 |
| 11h | C11/C13 alkyl (unbranched) | C11/C13 alkyl (unbranched) | 1:2 |
| 11i | C11/C13 alkyl (unbranched) | C15/C17 alkyl (unbranched) | 2:1 |
| 11j | C11/C13 alkyl (unbranched) | —(CH₂)₇CH═CH₂ | 1:1 |
| 11k | C11/C13 alkyl (unbranched) | —(CH₂)₇CH═CHCH₂CH₃ | 1:2 |
| 11l | C11/C13 alkyl (unbranched) | the R⁴—C═O residue in Formula (II) is | 2:1 |
| | | cocoyl | |
| 11m | C15/C17 alkyl (unbranched) | C7/C9 alkyl (unbranched) | 1:1 |
| 11n | C15/C17 alkyl (unbranched) | C11/C13 alkyl (unbranched) | 1:2 |
| 11o | C15/C17 alkyl (unbranched) | C15/C17 alkyl (unbranched) | 2:1 |
| 11p | C15/C17 alkyl (unbranched) | —(CH₂)₇CH═CH₂ | 1:1 |
| 11q | C15/C17 alkyl (unbranched) | —(CH₂)₇CH═CHCH₂CH₃ | 1:2 |
| 11r | C15/C17 alkyl (unbranched) | the R⁴—C═O residue in Formula (II) is | 2:1 |
| | | cocoyl | |
| 11s | —(CH₂)₇CH═CH₂ | C7/C9 alkyl (unbranched) | 1:1 |
| 11t | —(CH₂)₇CH═CH₂ | C11/C13 alkyl (unbranched) | 1:2 |
| 11u | —(CH₂)₇CH═CH₂ | C15/C17 alkyl (unbranched) | 2:1 |
| 11v | —(CH₂)₇CH═CH₂ | —(CH₂)₇CH═CH₂ | 1:1 |
| 11w | —(CH₂)₇CH═CH₂ | —(CH₂)₇CH═CHCH₂CH₃ | 1:2 |
| 11x | —(CH₂)₇CH═CH₂ | the R⁴—C═O residue in Formula (II) is | 2:1 |
| | | cocoyl | |
| 11y | —(CH₂)₇CH═CHCH₂CH₃ | C7/C9 alkyl (unbranched) | 1:1 |
| 11z | —(CH₂)₇CH═CHCH₂CH₃ | C11/C13 alkyl (unbranched) | 1:2 |
| 11aa | —(CH₂)₇CH═CHCH₂CH₃ | C15/C17 alkyl (unbranched) | 2:1 |
| 11bb | —(CH₂)₇CH═CHCH₂CH₃ | —(CH₂)₇CH═CH₂ | 1:1 |
| 11cc | —(CH₂)₇CH═CHCH₂CH₃ | —(CH₂)₇CH═CHCH₂CH₃ | 1:2 |
| 11dd | —(CH₂)₇CH═CHCH₂CH₃ | the R⁴—C═O residue in Formula (II) is | 2:1 |
| | | cocoyl | |
| 11ee | the R²—C═O residue in Formula (I) is | C7/C9 alkyl (unbranched) | 1:1 |
| | cocoyl | | |
| 11ff | the R²—C═O residue in Formula (I) is | C11/C13 alkyl (unbranched) | 1:2 |
| | cocoyl | | |
| 11gg | the R²—C═O residue in Formula (I) is | C15/C17 alkyl (unbranched) | 2:1 |
| | cocoyl | | |
| 11hh | the R²—C═O residue in Formula (I) is | —(CH₂)₇CH═CH₂ | 1:1 |
| | cocoyl | | |
| 11ii | the R²—C═O residue in Formula (I) is | —(CH₂)₇CH═CHCH₂CH₃ | 1:2 |
| | cocoyl | | |
| 11jj | the R²—C═O residue in Formula (I) is | the R⁴—C═O residue in Formula (II) is | 2:1 |
| | cocoyl | cocoyl | |

### Example 12—Example Mixtures of the Present Invention

Mixtures of a compound of Formula (I) and a compound of Formula (V) wherein the weight ratio of the compound of Formula (I) to the compound of Formula (V) is as indicated in Table 3.

TABLE 3

| | Compound of Formula (I) wherein | Compound of Formula (V) wherein | |
|---|---|---|---|
| | R¹ is methyl; and R² is as | n is 4; R⁹ is methyl; and R¹⁰ is as | |
| | indicated in this column | indicated in this column | |
| | 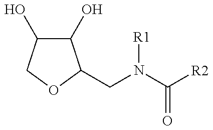 | 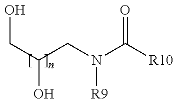 | |
| Example | (derived from xylose) | (derived from glucose) | Weight ratio |
| 12a | C7 alkyl (unbranched) | C7 alkyl (unbranched) | 1:1 |
| 12b | C7 alkyl (unbranched) | C9 alkyl (unbranched) | 1:2 |
| 12c | C7 alkyl (unbranched) | C11 alkyl (unbranched) | 2:1 |
| 12d | C7 alkyl (unbranched) | C13 alkyl (unbranched) | 1:1 |
| 12e | C7 alkyl (unbranched) | C15 alkyl (unbranched) | 1:2 |
| 12f | C7 alkyl (unbranched) | C17 alkyl (unbranched) | 2:1 |
| 12g | C9 alkyl (unbranched) | C7 alkyl (unbranched) | 1:1 |
| 12h | C9 alkyl (unbranched) | C9 alkyl (unbranched) | 1:2 |
| 12i | C9 alkyl (unbranched) | C11 alkyl (unbranched) | 2:1 |
| 12j | C9 alkyl (unbranched) | C13 alkyl (unbranched) | 1:1 |
| 12k | C9 alkyl (unbranched) | C15 alkyl (unbranched) | 1:2 |
| 12l | C9 alkyl (unbranched) | C17 alkyl (unbranched) | 2:1 |
| 12m | C11 alkyl (unbranched) | C7 alkyl (unbranched) | 1:1 |
| 12n | C11 alkyl (unbranched) | C9 alkyl (unbranched) | 1:2 |
| 12o | C11 alkyl (unbranched) | C11 alkyl (unbranched) | 2:1 |
| 12p | C11 alkyl (unbranched) | C13 alkyl (unbranched) | 1:1 |
| 12q | C11 alkyl (unbranched) | C15 alkyl (unbranched) | 1:2 |
| 12r | C11 alkyl (unbranched) | C17 alkyl (unbranched) | 2:1 |
| 12s | C13 alkyl (unbranched) | C7 alkyl (unbranched) | 1:1 |
| 12t | C13 alkyl (unbranched) | C9 alkyl (unbranched) | 1:2 |
| 12u | C13 alkyl (unbranched) | C11 alkyl (unbranched) | 2:1 |
| 12v | C13 alkyl (unbranched) | C13 alkyl (unbranched) | 1:1 |
| 12w | C13 alkyl (unbranched) | C15 alkyl (unbranched) | 1:2 |
| 12x | C13 alkyl (unbranched) | C17 alkyl (unbranched) | 2:1 |
| 12y | C15 alkyl (unbranched) | C7 alkyl (unbranched) | 1:1 |
| 12z | C15 alkyl (unbranched) | C9 alkyl (unbranched) | 1:2 |
| 12aa | C15 alkyl (unbranched) | C11 alkyl (unbranched) | 2:1 |
| 12bb | C15 alkyl (unbranched) | C13 alkyl (unbranched) | 1:1 |
| 12cc | C15 alkyl (unbranched) | C15 alkyl (unbranched) | 1:2 |
| 12dd | C15 alkyl (unbranched) | C17 alkyl (unbranched) | 2:1 |
| 12ee | C17 alkyl (unbranched) | C7 alkyl (unbranched) | 1:1 |
| 12ff | C17 alkyl (unbranched) | C9 alkyl (unbranched) | 1:2 |
| 12gg | C17 alkyl (unbranched) | C11 alkyl (unbranched) | 2:1 |
| 12hh | C17 alkyl (unbranched) | C13 alkyl (unbranched) | 1:1 |
| 12ii | C17 alkyl (unbranched) | C15 alkyl (unbranched) | 1:2 |
| 12jj | C17 alkyl (unbranched) | C17 alkyl (unbranched) | 2:1 |

### Example 13—Example Mixtures of the Present Invention

Mixtures of a compound of Formula (I) and a compound of Formula (V) wherein the weight ratio of the compound of Formula (I) to the compound of Formula (V) is as indicated in Table 4.

TABLE 4

| | Compound of Formula (I) | Compound of Formula (V) wherein | |
|---|---|---|---|
| | wherein R¹ is methyl; and R² is | n is 4; R⁹ is methyl; and R¹⁰ is as | |
| | indicated in this column | indicated in this column | |
| | 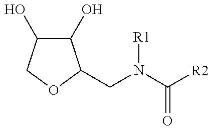 | 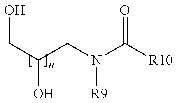 | |
| Example | (derived from xylose) | (derived from glucose) | Weight ratio |
| 13a | C7/C9 alkyl (unbranched) | C7/C9 alkyl (unbranched) | 1:1 |
| 13b | C7/C9 alkyl (unbranched) | C11/C13 alkyl (unbranched) | 1:2 |
| 13c | C7/C9 alkyl (unbranched) | C15/C17 alkyl (unbranched) | 2:1 |
| 13d | C7/C9 alkyl (unbranched) | —(CH₂)₇CH═CH₂ | 1:1 |
| 13e | C7/C9 alkyl (unbranched) | —(CH₂)₇CH═CHCH₂CH₃ | 1:2 |
| 13f | C7/C9 alkyl (unbranched) | the R¹⁰—C═O residue in Formula (II) is | 2:1 |
| | | cocoyl | |
| 13g | C11/C13 alkyl (unbranched) | C7/C9 alkyl (unbranched) | 1:1 |
| 13h | C11/C13 alkyl (unbranched) | C11/C13 alkyl (unbranched) | 1:2 |
| 13i | C11/C13 alkyl (unbranched) | C15/C17 alkyl (unbranched) | 2:1 |
| 13j | C11/C13 alkyl (unbranched) | —(CH₂)₇CH═CH₂ | 1:1 |
| 13k | C11/C13 alkyl (unbranched) | —(CH₂)₇CH═CHCH₂CH₃ | 1:2 |
| 13l | C11/C13 alkyl (unbranched) | the R¹⁰—C═O residue in Formula (II) is | 2:1 |
| | | cocoyl | |
| 13m | C15/C17 alkyl (unbranched) | C7/C9 alkyl (unbranched) | 1:1 |
| 13n | C15/C17 alkyl (unbranched) | C11/C13 alkyl (unbranched) | 1:2 |
| 13o | C15/C17 alkyl (unbranched) | C15/C17 alkyl (unbranched) | 2:1 |
| 13p | C15/C17 alkyl (unbranched) | —(CH₂)₇CH═CH₂ | 1:1 |
| 13q | C15/C17 alkyl (unbranched) | —(CH₂)₇CH═CHCH₂CH₃ | 1:2 |
| 13r | C15/C17 alkyl (unbranched) | the R¹⁰—C═O residue in Formula (II) is | 2:1 |
| | | cocoyl | |
| 13s | —(CH₂)₇CH═CH₂ | C7/C9 alkyl (unbranched) | 1:1 |
| 13t | —(CH₂)₇CH═CH₂ | C11/C13 alkyl (unbranched) | 1:2 |
| 13u | —(CH₂)₇CH═CH₂ | C15/C17 alkyl (unbranched) | 2:1 |
| 13v | —(CH₂)₇CH═CH₂ | —(CH₂)₇CH═CH₂ | 1:1 |
| 13w | —(CH₂)₇CH═CH₂ | —(CH₂)₇CH═CHCH₂CH₃ | 1:2 |
| 13x | —(CH₂)₇CH═CH₂ | the R¹⁰—C═O residue in Formula (II) is | 2:1 |
| | | cocoyl | |
| 13y | —(CH₂)₇CH═CHCH₂CH₃ | C7/C9 alkyl (unbranched) | 1:1 |
| 13z | —(CH₂)₇CH═CHCH₂CH₃ | C11/C13 alkyl (unbranched) | 1:2 |
| 13aa | —(CH₂)₇CH═CHCH₂CH₃ | C15/C17 alkyl (unbranched) | 2:1 |
| 13bb | —(CH₂)₇CH═CHCH₂CH₃ | —(CH₂)₇CH═CH₂ | 1:1 |
| 13cc | —(CH₂)₇CH═CHCH₂CH₃ | —(CH₂)₇CH═CHCH₂CH₃ | 1:2 |
| 13dd | —(CH₂)₇CH═CHCH₂CH₃ | the R¹⁰—C═O residue in Formula (II) is | 2:1 |
| | | cocoyl | |
| 13ee | the R²—C═O residue in Formula (I) is | C7/C9 alkyl (unbranched) | 1:1 |
| | cocoyl | | |
| 13ff | the R²—C═O residue in Formula (I) is | C11/C13 alkyl (unbranched) | 1:2 |
| | cocoyl | | |
| 13gg | the R²—C═O residue in Formula (I) is | C15/C17 alkyl (unbranched) | 2:1 |
| | cocoyl | | |
| 13hh | the R²—C═O residue in Formula (I) is | —(CH₂)₇CH═CH₂ | 1:1 |
| | cocoyl | | |
| 13ii | the R²—C═O residue in Formula (I) is | —(CH₂)₇CH═CHCH₂CH₃ | 1:2 |
| | cocoyl | | |
| 13jj | the R²—C═O residue in Formula (I) is | the R¹⁰—C═O residue in Formula (II) is | 2:1 |
| | cocoyl | cocoyl | |

### Example 14—Example Mixtures of the Present Invention

Mixtures of a compound of Formula (II) and a compound of Formula (V) wherein the weight ratio of the compound of Formula (II) to the compound of Formula (V) is as indicated in Table 5.

TABLE 5

| | Compound of Formula (II) wherein | Compound of Formula (V) wherein | |
|---|---|---|---|
| | R³ is methyl; and R⁴ is as | n is 4; R⁹ is methyl; and R¹⁰ is as | |
| | indicated in this column | indicated in this column | |
| | 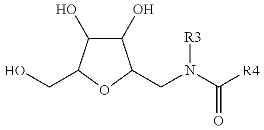 | 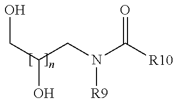 | |
| Example | (derived from glucose) | (derived from glucose) | Weight ratio |
| 14a | C7 alkyl (unbranched) | C7 alkyl (unbranched) | 1:1 |
| 14b | C7 alkyl (unbranched) | C9 alkyl (unbranched) | 1:2 |
| 14c | C7 alkyl (unbranched) | C11 alkyl (unbranched) | 2:1 |
| 14d | C7 alkyl (unbranched) | C13 alkyl (unbranched) | 1:1 |
| 14e | C7 alkyl (unbranched) | C15 alkyl (unbranched) | 1:2 |
| 14f | C7 alkyl (unbranched) | C17 alkyl (unbranched) | 2:1 |
| 14g | C9 alkyl (unbranched) | C7 alkyl (unbranched) | 1:1 |
| 14h | C9 alkyl (unbranched) | C9 alkyl (unbranched) | 1:2 |
| 14i | C9 alkyl (unbranched) | C11 alkyl (unbranched) | 2:1 |
| 14j | C9 alkyl (unbranched) | C13 alkyl (unbranched) | 1:1 |
| 14k | C9 alkyl (unbranched) | C15 alkyl (unbranched) | 1:2 |
| 14l | C9 alkyl (unbranched) | C17 alkyl (unbranched) | 2:1 |
| 14m | C11 alkyl (unbranched) | C7 alkyl (unbranched) | 1:1 |
| 14n | C11 alkyl (unbranched) | C9 alkyl (unbranched) | 1:2 |
| 14o | C11 alkyl (unbranched) | C11 alkyl (unbranched) | 2:1 |
| 14p | C11 alkyl (unbranched) | C13 alkyl (unbranched) | 1:1 |
| 14q | C11 alkyl (unbranched) | C15 alkyl (unbranched) | 1:2 |
| 14r | C11 alkyl (unbranched) | C17 alkyl (unbranched) | 2:1 |
| 14s | C13 alkyl (unbranched) | C7 alkyl (unbranched) | 1:1 |
| 14t | C13 alkyl (unbranched) | C9 alkyl (unbranched) | 1:2 |
| 14u | C13 alkyl (unbranched) | C11 alkyl (unbranched) | 2:1 |
| 14v | C13 alkyl (unbranched) | C13 alkyl (unbranched) | 1:1 |
| 14w | C13 alkyl (unbranched) | C15 alkyl (unbranched) | 1:2 |
| 14x | C13 alkyl (unbranched) | C17 alkyl (unbranched) | 2:1 |
| 14y | C15 alkyl (unbranched) | C7 alkyl (unbranched) | 1:1 |
| 14z | C15 alkyl (unbranched) | C9 alkyl (unbranched) | 1:2 |
| 14aa | C15 alkyl (unbranched) | C11 alkyl (unbranched) | 2:1 |
| 14bb | C15 alkyl (unbranched) | C13 alkyl (unbranched) | 1:1 |
| 14cc | C15 alkyl (unbranched) | C15 alkyl (unbranched) | 1:2 |
| 14dd | C15 alkyl (unbranched) | C17 alkyl (unbranched) | 2:1 |
| 14ee | C17 alkyl (unbranched) | C7 alkyl (unbranched) | 1:1 |
| 14ff | C17 alkyl (unbranched) | C9 alkyl (unbranched) | 1:2 |
| 14gg | C17 alkyl (unbranched) | C11 alkyl (unbranched) | 2:1 |
| 14hh | C17 alkyl (unbranched) | C13 alkyl (unbranched) | 1:1 |
| 14ii | C17 alkyl (unbranched) | C15 alkyl (unbranched) | 1:2 |
| 14jj | C17 alkyl (unbranched) | C17 alkyl (unbranched) | 2:1 |

### Example 15—Example Mixtures of the Present Invention

Mixtures of a compound of Formula (II) and a compound of Formula (V) wherein the weight ratio of the compound of Formula (II) to the compound of Formula (V) is as indicated in Table 6.

TABLE 6

| | Compound of Formula (II) wherein | Compound of Formula (V) wherein | |
|---|---|---|---|
| | R³ is methyl; and R⁴ is as | n is 4; R⁹ is methyl; and R¹⁰ is as | |
| | indicated in this column | indicated in this column | |
| | 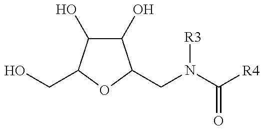 | 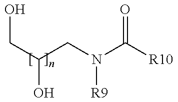 | |
| Example | (derived from glucose) | (derived from glucose) | Weight ratio |
| 15a | C7/C9 alkyl (unbranched) | C7/C9 alkyl (unbranched) | 1:1 |
| 15b | C7/C9 alkyl (unbranched) | C11/C13 alkyl (unbranched) | 1:2 |
| 15c | C7/C9 alkyl (unbranched) | C15/C17 alkyl (unbranched) | 2:1 |
| 15d | C7/C9 alkyl (unbranched) | —(CH₂)₇CH═CH₂ | 1:1 |
| 15e | C7/C9 alkyl (unbranched) | —(CH₂)₇CH═CHCH₂CH₃ | 1:2 |
| 15f | C7/C9 alkyl (unbranched) | the R¹⁰—C═O residue in Formula (II) is | 2:1 |
| | | cocoyl | |
| 15g | C11/C13 alkyl (unbranched) | C7/C9 alkyl (unbranched) | 1:1 |
| 15h | C11/C13 alkyl (unbranched) | C11/C13 alkyl (unbranched) | 1:2 |
| 15i | C11/C13 alkyl (unbranched) | C15/C17 alkyl (unbranched) | 2:1 |
| 15j | C11/C13 alkyl (unbranched) | —(CH₂)₇CH═CH₂ | 1:1 |
| 15k | C11/C13 alkyl (unbranched) | —(CH₂)₇CH═CHCH₂CH₃ | 1:2 |
| 15l | C11/C13 alkyl (unbranched) | the R¹⁰—C═O residue in Formula (II) is | 2:1 |
| | | cocoyl | |
| 15m | C15/C17 alkyl (unbranched) | C7/C9 alkyl (unbranched) | 1:1 |
| 15n | C15/C17 alkyl (unbranched) | C11/C13 alkyl (unbranched) | 1:2 |
| 15o | C15/C17 alkyl (unbranched) | C15/C17 alkyl (unbranched) | 2:1 |
| 15p | C15/C17 alkyl (unbranched) | —(CH₂)₇CH═CH₂ | 1:1 |
| 15q | C15/C17 alkyl (unbranched) | —(CH₂)₇CH═CHCH₂CH₃ | 1:2 |
| 15r | C15/C17 alkyl (unbranched) | the R¹⁰—C═O residue in Formula (II) is | 2:1 |
| | | cocoyl | |
| 15s | —(CH₂)₇CH═CH₂ | C7/C9 alkyl (unbranched) | 1:1 |
| 15t | —(CH₂)₇CH═CH₂ | C11/C13 alkyl (unbranched) | 1:2 |
| 15u | —(CH₂)₇CH═CH₂ | C15/C17 alkyl (unbranched) | 2:1 |
| 15v | —(CH₂)₇CH═CH₂ | —(CH₂)₇CH═CH₂ | 1:1 |
| 15w | —(CH₂)₇CH═CH₂ | —(CH₂)₇CH═CHCH₂CH₃ | 1:2 |
| 15x | —(CH₂)₇CH═CH₂ | the R¹⁰—C═O residue in Formula (II) is | 2:1 |
| | | cocoyl | |
| 15y | —(CH₂)₇CH═CHCH₂CH₃ | C7/C9 alkyl (unbranched) | 1:1 |
| 15z | —(CH₂)₇CH═CHCH₂CH₃ | C11/C13 alkyl (unbranched) | 1:2 |
| 15aa | —(CH₂)₇CH═CHCH₂CH₃ | C15/C17 alkyl (unbranched) | 2:1 |
| 15bb | —(CH₂)₇CH═CHCH₂CH₃ | —(CH₂)₇CH═CH₂ | 1:1 |
| 15cc | —(CH₂)₇CH═CHCH₂CH₃ | —(CH₂)₇CH═CHCH₂CH₃ | 1:2 |
| 15dd | —(CH₂)₇CH═CHCH₂CH₃ | the R¹⁰—C═O residue in Formula (II) is | 2:1 |
| | | cocoyl | |
| 15ee | the R⁴—C═O residue in Formula (I) is | C7/C9 alkyl (unbranched) | 1:1 |
| | cocoyl | | |
| 15ff | the R⁴—C═O residue in Formula (I) is | C11/C13 alkyl (unbranched) | 1:2 |
| | cocoyl | | |
| 15gg | the R⁴—C═O residue in Formula (I) is | C15/C17 alkyl (unbranched) | 2:1 |
| | cocoyl | | |
| 15hh | the R⁴—C═O residue in Formula (I) is | —(CH₂)₇CH═CH₂ | 1:1 |
| | cocoyl | | |
| 15ii | the R⁴—C═O residue in Formula (I) is | —(CH₂)₇CH═CHCH₂CH₃ | 1:2 |
| | cocoyl | | |
| 15jj | the R⁴—C═O residue in Formula (I) is | the R¹⁰—C═O residue in Formula (II) is | 2:1 |
| | cocoyl | cocoyl | |

### Example 16—Example Mixtures of the Present Invention

| Example | Mixture |
|---|---|
| 16a | 50 wt.- % of a mixture of any of Examples |
| | 10a to 10jj (Table 1) + 50 wt.-% water |
| 16b | 50 wt.- % of a mixture of any of Examples |
| | 11a to 11jj (Table 2) + 50 wt.-% water |
| 16c | 50 wt.- % of a mixture of any of Examples |
| | 12a to 12jj (Table 3) + 50 wt.-% water |
| 16d | 50 wt.- % of a mixture of any of Examples |
| | 13a to 13jj (Table 4) + 50 wt.-% water |
| 16e | 50 wt.- % of a mixture of any of Examples |
| | 14a to 14jj (Table 5) + 50 wt.-% water |
| 16f | 50 wt.- % of a mixture of any of Examples |
| | 15a to 15jj (Table 6) + 50 wt.-% water |

### Example 17—Example Formulations of the Present Invention

| | Example | | | | |
|---|---|---|---|---|---|
| | 17a | 17b | 17c | 17d | 17e |
| Formulation | Liquid | Body wash | Wet wipe | Shampoo | Shampoo |
| | soap | | lotion | | |
| Any of the mixtures of | — | — | — | — | 1.0 |
| Table 1 | | | | | |
| Any of the mixtures of | — | — | — | 1.5 | — |
| Table 4 | | | | | |
| Cyclic N- | 1.5 | 1.0 | 1.0 | — | — |
| Octanoyl/Decanoyl-N- | | | | | |
| Methylxylosamide | | | | | |
| Phenoxyethanol | 1.0 | — | 1.0 | — | 1.0 |
| Benzoic acid | — | 0.5 | — | 0.5 | — |
| SLES | 10 | — | — | — | 9 |
| Cocamidopropylbetaine | 2 | 4.5 | — | — | 2 |
| Cocamide MEA | — | — | — | — | 1 |
| Sodium Cocoyl | — | 2 | 1 | 2 | — |
| Glutamate | | | | | |
| Sodium Cocoyl | — | 2 | 1 | 2 | — |
| Glycinate | | | | | |
| Sodium Cocoyl | — | 2 | — | — | — |
| Isethionate | | | | | |
| Cocoyl Methyl | — | 3 | 2 | — | — |
| Glucamide | | | | | |
| EGDS | — | 0.5 | — | — | 0.7 |
| PEG-7 Glyceryl | — | — | 1 | — | — |
| Cocoate | | | | | |
| Cocoyl Glucoside | — | — | — | 5 | — |
| Lauryl Glucoside | — | — | — | 5 | — |
| Acrylates Copolymer | — | — | — | 2 | — |
| Glycerine | — | — | — | 1 | 2 |
| Panthenol | — | — | — | 0.2 | 0.2 |
| Dimethicone | — | — | — | — | — |
| Polyquaternium 7 (PQ-7) | — | — | — | — | 0.6 |
| Guar | — | — | — | — | 0.3 |
| Hydroxypropyltrimonium | | | | | |
| Chloride | | | | | |
| Hydrolyzed Protein | — | — | — | — | 0.1 |
| Perfume | 0.2 | 0.2 | 0.1 | — | 0.8 |
| NaCl | 1.5 | 1.0 | 0.5 | 0.3 | 1.5 |
| Water | QSP | QSP | QSP | QSP | QSP |
| Total | 100% | 100% | 100% | 100% | 100% |

### Example 18—Example Formulations of the Present Invention

| | Example | | | | |
|---|---|---|---|---|---|
| | 18a | 18b | 18c | 18d | 18e |
| Formulation | § | § | § | § | § |
| Any of the mixtures of Table | — | — | — | — | 1.0 |
| 1 | | | | | |
| Any of the mixtures of Table | — | — | — | 1.5 | — |
| 2 | | | | | |
| Any of the mixtures of Table | — | 1.0 | — | — | — |
| 3 | | | | | |
| Cyclic N-Octanoyl/Decanoyl- | 1.0 | — | 1.0 | — | — |
| N-Methylxylosamide | | | | | |
| Benzoic acid | 0.5 | — | 0.5 | — | 0.5 |
| Phenoxyethanol | — | 1.0 | — | 1.0 | — |
| Sodium Lauryl Ether | 18 | 18 | 19 | 8 | — |
| Sulfate (SLES) | | | | | |
| Cocamidopropylbetaine | 8 | — | — | 4 | — |
| Sodium Alkane Sulfonate | — | — | — | 24 | — |
| Lauryldimethylamine | — | — | 6 | — | 5 |
| oxide | | | | | |
| Ethanol | 7 | 4 | — | — | — |
| Coco-Glucamide | — | 8 | — | — | — |
| Perfume | 0.2 | 0.2 | 0.1 | 0.1 | 0.8 |
| Cumene Sulfonate | — | — | 1.5 | — | — |
| Alpha-Olefine Sulfonate | — | — | — | — | 15 |
| (Na salt) | | | | | |
| Water & Auxiliaries | QSP | QSP | QSP | QSP | QSP |
| Total | 100% | 100% | 100% | 100% | 100% |

KEY: § = hand dishwashing formulation.

### Example 19—Example Formulations of the Present Invention

| | Example | | | |
|---|---|---|---|---|
| | | 19b | 19c | |
| | | Intensive | Anti- | |
| | 19a | hand and | ageing | 19d |
| | Body | body | night | Baby |
| Formulation | lotion | lotion | cream | cream |
| Any of the mixtures of Table 2 | — | — | — | 1.5 |
| Any of the mixtures of Table 4 | — | 1.0 | — | — |
| Cyclic N-Octanoyl/Decanoyl-N- | 1.5 | — | 1.0 | — |
| Methylxylosamide | | | | |
| Phenoxyethanol | 1.0 | — | 1.0 | — |
| Benzoic acid | — | 0.5 | — | 0.5 |
| Ethylhexyl Stearate | 7 | — | — | — |
| Decyl Oleate | 5 | — | — | — |
| Plantasens ® | 3 | — | — | — |
| Natural Emulsifier HE | | | | |
| 20 (Clariant), which is Cetearyl | | | | |
| Glucoside (and) Sorbitan Olivate | | | | |
| Dimethicone | 2 | — | — | — |
| Glycerine | 3 | 3 | 7 | — |
| Xanthan Gum | 0.2 | — | — | — |
| Aristoflex ® AVC from Clariant | 0.5 | 1 | — | — |
| (Ammonium | | | | |
| Acryloyldimethyltaurate/ | | | | |
| VP Copolymer) | | | | |
| Fragrance | 0.3 | — | 0.3 | 0.3 |
| Polyglyceryl-2- | — | 0.5 | — | 2 |
| Sesquiisostearate | | | | |
| Trilaureth-4 Phosphate | — | 2 | — | — |
| Mineral Oil (Paraffinum Liquidum) | — | 5 | — | — |
| Plantasens ® Refined Organic | — | 2 | — | — |
| Babassu Butter from Clariant | | | | |
| (Orbignya Oleifera Seed Oil) | | | | |
| Squalane | — | 2 | 12 | — |
| Macadamia Ternifolia Seed Oil | — | 2 | — | — |
| (and) Crambe Abyssinica Seed | | | | |
| Oil (and) Orbignya Oleifera Seed | | | | |
| Oil | | | | |
| Silcare ® Silicone | — | 0.5 | — | — |
| SEA from Clariant | | | | |
| (Trideceth-9 PG-Amodimethicone | | | | |
| and Trideceth-12) | | | | |
| Cetearyl Alcohol | — | 2 | — | — |
| Isopropyl Palmitate | — | 4 | — | — |
| Dow Corning ® 345 | — | — | 10 | — |
| (Cyclopentasiloxane and | | | | |
| Cyclohexasiloxane) | | | | |
| Paraffin Oil, low viscosity | — | — | 4 | 10 |
| Petrolatum | — | — | 4 | 15 |
| Cetyl Alcohol | — | — | 3 | — |
| Cutina ® GMS (Glyceryl Stearate) | — | — | 2.5 | — |
| PEG-40 Stearate | — | — | 3 | — |
| Cera Alba Wax | — | — | 2 | — |
| Mangifera Indica (Mango) Seed | — | — | 2 | — |
| Butter | | | | |
| Abyssinian Oil (and) Phytosterols | — | — | 0.5 | — |
| (and) Olea Europea (Olive) Oil | | | | |
| Unsaponifiables | | | | |
| Sorbitan Tristearate | — | — | 0.3 | — |
| Ubiquinone | — | — | 0.05 | — |
| Aristoflex ® Velvet from Clariant | — | — | 0.3 | — |
| (Polyacrylate Crosspolymer-11) | | | | |
| Hostacerin ® SFO from Clariant | — | — | — | 3.5 |
| (Sunflower Seed Oil Sorbitol | | | | |
| Esters) | | | | |
| Paracera ® M | — | — | — | 2 |
| (Cera Microcristallina) | | | | |
| Beeswax | — | — | — | 1 |
| Magnesium Stearate | — | — | — | 1 |
| Talc | — | — | — | 10 |
| Zinc Oxide | — | — | — | 10 |
| Extrapon Hamamelis | — | — | — | 2 |
| D-Panthenol | — | — | — | 2 |
| Water | QSP | QSP | QSP | QSP |
| Total | 100% | 100% | 100% | 100% |

### Example 20—Example Formulations of the Present Invention

| | Example | | | | |
|---|---|---|---|---|---|
| | 20a | 20b | 20c | 20d | 20e |
| Formulation | $ | $ | $ | $ | $ |
| Any of the mixtures of Table 1 | — | — | — | 1.0 | 1.5 |
| Any of the mixtures of Table 3 | — | — | 1.5 | — | — |
| Cyclic N-Octanoyl/Decanoyl- | 1.0 | 1.0 | — | — | — |
| N-Methylxylosamide | | | | | |
| Benzoic acid | 0.5 | — | 0.5 | — | 0.5 |
| Phenoxyethanol | — | 1.0 | — | 1.0 | — |
| Sodium Lauryl Ether | 2 | 2 | 2 | — | — |
| Sulfate (SLES) | | | | | |
| Lauryldimethylamine | 2 | — | — | — | 2 |
| Oxide | | | | | |
| Alkylbenzenesulfonate | — | — | — | 2 | 2 |
| Laureth-7 | — | — | 2 | 2 | — |
| Capric/Caprylic-Glucamide | — | 2 | — | — | — |
| Perfume | 0.2 | 0.2 | 0.1 | 0.1 | 0.8 |
| Water & Auxiliaries | QSP | QSP | QSP | QSP | QSP |
| Total | 100% | 100% | 100% | 100% | 100% |

KEY: § = hard surface cleaner

### Example 21—Example Formulations of the Present Invention

| | Example | | | | |
|---|---|---|---|---|---|
| | 21a | 21b | 21c | 21d | 21e |
| Formulation | & | & | & | & | & |
| Any of the mixtures of Table 2 | — | 1.0 | — | 1.5 | 1.5 |
| Cyclic N-Octanoyl/Decanoyl- | 1.0 | — | 1.0 | — | — |
| N-Methylxylosamide | | | | | |
| Phenoxyethanol | 1.0 | — | 1.0 | — | 1.0 |
| Benzoic acid | — | 0.5 | — | 0.5 | — |
| Sodium Lauryl Ether | 10 | 15 | 5 | 15 | 2 |
| Sulfate (SLES) | | | | | |
| Alkylbenzenesulfonate | 10 | 5 | 15 | 0 | 15 |
| Laureth-7 | 10 | 10 | 10 | 10 | 8 |
| C₁₂/C₁₄ Fatty Acid Soap | 3 | 3 | 3 | 5 | 5 |
| Perfume | 0.2 | 0.2 | 0.1 | 0.1 | 0.8 |
| Water & Auxiliaries | QSP | QSP | QSP | QSP | QSP |
| Total | 100% | 100% | 100% | 100% | 100% |

KEY: & = liquid laundry detergent formulation

### Example 22—Example Formulations of the Present Invention

| | Example | | | | | |
|---|---|---|---|---|---|---|
| | 22a | 22b | 22c | 22d | 22e | 22f |
| Formulation | Soap | Soap | Body | Shampoo | Wash | Body |
| | formulation | bar | wash | | cream | wash |
| Any of the mixtures of Table | — | — | — | — | 1.0 | — |
| 1 | | | | | | |
| Any of the mixtures of Table | — | — | — | 1.5 | — | 0.8 |
| 2 | | | | | | |
| Any of the mixtures of Table | — | 1.5 | — | — | — | — |
| 3 | | | | | | |
| Cyclic N-Octanoyl/Decanoyl- | 1.5 | — | 1.0 | — | — | — |
| N-Methylxylosamide | | | | | | |
| Benzoic acid | 0.5 | — | 0.5 | — | 0.5 | — |
| Phenoxyethanol | — | 1.0 | — | 1.0 | — | 1.0 |
| Sodium Lauryl Sulfate | — | — | 10 | — | — | — |
| Ammonium Lauryl Sulfate | — | — | — | 12 | — | — |
| Sodium Lauryl Ether Sulfate | 15 | — | 2 | — | — | — |
| (SLES) | | | | | | |
| Cocamidopropylbetaine | 7 | — | 2 | 3 | — | 2 |
| Cocamide MEA | — | — | 1 | — | — | 2 |
| Lauric Acid | 0.5 | 2 | — | — | — | 10 |
| Myristic Acid | 1.5 | 2 | — | — | — | 10 |
| Stearic Acid | 0.5 | 2 | — | — | 10 | — |
| Palmitic Acid | — | 2 | — | — | — | 10 |
| Potassium Tallowate | — | 2 | — | — | — | — |
| Sodium Palm Kernelate | — | 20 | — | — | — | — |
| Sodium Cocoate | 1 | 60 | — | — | — | — |
| Lauryl Glucoside | — | — | — | 4 | — | — |
| Sodium Cocoyl Isethionate | — | — | — | — | 14 | — |
| Glycerine | — | 5 | — | — | — | 5 |
| Cetearyl Alcohol | 1.5 | — | — | — | 2 | — |
| Dimethicone | — | — | — | — | 0.1 | — |
| Polyquaternium 7 (PQ-7) | — | — | — | 0.2 | — | — |
| Guar | — | — | 0.3 | — | — | — |
| Hydroxypropyltrimonium | | | | | | |
| Chloride | | | | | | |
| Hydrolyzed Protein | — | — | — | 0.1 | — | — |
| Perfume | 1 | 0.2 | 0.1 | — | 0.8 | 0.7 |
| NaCl | 1.5 | — | 0.5 | 0.3 | — | 2.5 |
| Water | QSP | QSP | QSP | QSP | QSP | QSP |
| Total | 100% | 100% | 100% | 100% | 100% | 100% |

### Example 23—MIC Data

For testing the inhibition of bacteria, the sample was diluted in demineralized water and added in different concentrations to liquid Caso-Agar at 50° C. and is buffered to pH 7 (+/−0.2). For testing the inhibition of yeast and mold, the sample was diluted in demineralized water and added in different concentrations (500 ppm, 1000 ppm, 1500 ppm, 2000 ppm, 3000 ppm, 4000 ppm, 5000 ppm, 7500 ppm, 10000 ppm and 15000 ppm) to liquid Sabouraud-4% Dextrose-Agar at 50° C. and is buffered to pH 5.6 (+/−0.2). Each of the solutions is poured into a petri-dish and inoculated with the same amount of bacteria, yeast or mold respectively. The minimum inhibitory concentration (MIC) is the lowest concentration of the sample that inhibits the growth of the respective microorganism where the next lowest dilution fails to inhibit the growth of said microorganism.

TABLE
MIC data

| Organisms | MIC of N-Methyl Xylosamid* |
|---|---|
| Bacteria (B), Yeast (Y) and Mold (M) | (70% active) in water [ppm] |
| Staphylococcus aureus (B) | <500 |
| Pseudomonas aeruginosa (B) | 10000 |
| Escherichia coli (B) | 15000 |
| Enterobacter aerogenes (B) | 15000 |
| Klebsiella pneumoniae (B) | 1000 |
| Burkholderia cepacia (B) | 7500 |
| Pluralibacter gergoviae (B) | 15000 |
| Serratia marcescens (B) | 10000 |
| Candida albicans (Y) | 3000 |
| Aspergillus brasiliensis (M) | 2000 |
| Penicillium minioluteum (M) | 1500 |
| Aspergillus terreus (M) | 3000 |
| Fusarium solani (M) | 1000 |
| Penicillium funicolosum (M) | 1000 |
| Saccharomyces cerevisiae (Y) | 1000 |
| Candida parapsilosis (Y) | 7500 |

*N-Methyl Xylosamid as used herein refers to a compound of Formula (I) wherein R1 is methyl and R2 is linear C7/C9 alkyl

The data shows excellent performance of the compound of Formula (I) towards various microorganisms, both bacteria and fungi.

1-17. (canceled)

18. A compound of Formula (I) 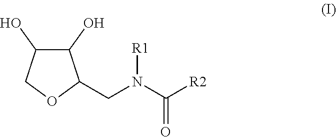
wherein
R¹ is selected from hydrogen, C1-C16 alkyl, C1-C3 hydroxyalkyl, and methoxy-(C1-C3)-alkyl; and
R² is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl.

19. The compound of Formula (I) according to claim 18, wherein
R¹ is selected from C1-C4 alkyl; and
R² is selected from C7-C21 alkyl, C7-C21 monoalkenyl, and C7-C21 dialkenyl.

20. An antimicrobial blend comprising
(A) a compound of Formula (I) as defined in claim 18, as component A; and
(B) an antimicrobial agent or a preservative booster, as component B.

21. The antimicrobial blend according to claim 20, wherein the weight ratio of component A to component B is from 1:20 to 20:1.

22. The antimicrobial blend according to claim 20, wherein the antimicrobial agent (component B) is selected from the group consisting of aromatic alcohols, organic acids and salts thereof, hydroxamic acids and salts thereof, hydroxypyridones, alkyl diols, halogenated compounds, isothiazolinones, and mixtures thereof.

23. The antimicrobial blend according to claim 20, wherein the preservative booster (component B) is selected from the group consisting of compounds of Formula (VIII) 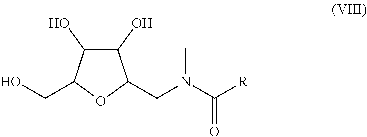
wherein R is selected from saturated hydrocarbon chains having 5 to 23 carbon atoms, unsaturated hydrocarbon chains having 5 to 23 carbon atoms, and mixtures thereof;
sorbitan esters; isosorbide esters; glyceryl ethers; and glyceryl esters.

24. A mixture comprising a first sugar amide and a second sugar amide, wherein the first sugar amide is a cyclic sugar amide.

25. The mixture according to claim 24, wherein the second sugar amide is a linear sugar amide or a cyclic sugar amide.

26. The mixture according to claim 24, wherein the first sugar amide is derived from glucose, xylose, arabinose, galactose, or mannose, and/or wherein the second sugar amide is derived from glucose, xylose, arabinose, galactose, or mannose.

27. The mixture according to claim 24, wherein the cyclic sugar amide is selected from:
a compound of Formula (I) 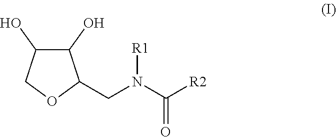
wherein
R¹ is selected from hydrogen, C1-C16 alkyl, C1-C3 hydroxyalkyl, and methoxy-(C1-C3)-alkyl; and
R² is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
a compound of Formula (II) 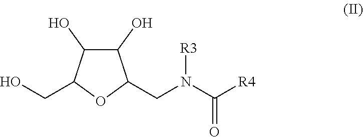
wherein
R³ is selected from hydrogen, C1-C16 alkyl, C1-C3 hydroxyalkyl, and methoxy-(C1-C3)-alkyl; and
R⁴ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
a compound of Formula (III) 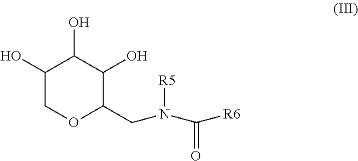
wherein
R⁵ is selected from hydrogen, C1-C16 alkyl, C1-C3 hydroxyalkyl, and methoxy-(C1-C3)-alkyl; and
R⁶ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl;
and
a compound of Formula (IV) 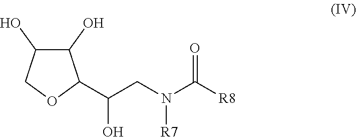
wherein
R⁷ is selected from hydrogen, C1-C16 alkyl, C1-C3 hydroxyalkyl, and methoxy-(C1-C3)-alkyl; and
R⁸ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl.

28. The mixture according to claim 24, wherein the linear sugar amide is a compound of Formula (V) 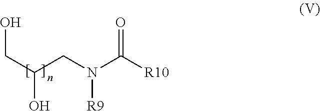
wherein
R⁹ is selected from hydrogen, C1-C16 alkyl, C1-C3 hydroxyalkyl, and methoxy-(C1-C3)-alkyl;
R¹⁰ is selected from C5-C23 alkyl, C5-C23 monoalkenyl, C5-C23 dialkenyl, C5-C23 trialkenyl, C5-C23 hydroxyalkyl, C5-C23 hydroxymonoalkenyl, C5-C23 hydroxydialkenyl, and C5-C23 hydroxytrialkenyl; and
n is selected from 1 to 4.

29. The mixture according to claim 24, wherein the weight ratio of the first sugar amide to the second sugar amide is from 99.5:0.5 to 0.5:99.5.

30. A formulation comprising from 0.001 to 99.999 wt.-% of the compound of Formula (I) according to claim 18 and one or more further components.

31. The formulation according to claim 30, wherein the formulation is a cosmetic formulation or a household cleaning formulation.

32. The formulation according to claim 30, wherein the formulation is selected from the group consisting of shampoo, body wash, facial cleanser, face mask, bubble bath, intimate wash, bath oil, cleansing milk, micellar water, make-up remover, cleansing wipes, hair mask, perfume, liquid soap, shaving soap, shaving foam, cleansing foam, day cream, anti-ageing cream, body milk, body lotion, body mousse, face serum, eye cream, sunscreen lotion, sun cream, face cream, after-shave lotion, pre-shaving cream, depilatory cream, skin-whitening gel, self-tanning cream, anti-acne gel, mascara, foundation, primer, concealer, blush, bronzer, blemish balm (bb) cream, eyeliner, night cream, eye brow gel, highlighter, lip stain, hand sanitizer, hair oil, nail varnish remover, conditioner, hair styling gel, hair styling cream, anti-frizz serum, scalp treatment, hair colorant, split end fluid, deodorant, antiperspirant, baby cream, insect repellent, hand cream, sunscreen gel, foot cream, exfoliator, body scrub, cellulite treatment, bar soap, cuticle cream, lip balm, hair treatment, eye shadow, bath additive, body mist, eau de toilette, mouthwash, toothpaste, lubricating gel, moisturizer, serum, toner, aqua sorbet, cream gel, styling mousse, dry shampoo, lip stick, lip gloss, hydro-alcoholic gel, body oil, shower milk, illuminator, lip crayon, hair spray, combing cream, and sunblock.
